SEQUENCE LISTING (20 sequences)

SEQ ID NO: 1 - DNA, 2283 bp, Arabidopsis thaliana
  CDS (86)..(2074)
agctttctccattttcacctctctctctctgtgttttttttaactccgtttaaattttccgtctgaattttacaaaggaacactaatgtcagcttttgacggcgttgaaaatcaaatgaacggtccagattcatctcctcgtctctctcaagatcctcgtgagcctcgctcattattatcatcttcttgtttcccaatcaccctcaagttcgttgatgtgtgttaccgagtcaagatccatggcatgagtaacgattcttgtaatatcaagaaactattaggattaaaacaaaaaccgtccgatgagacaagatcgacggaggagaggacgatactaagcggagtcaccggaatgatatcacccggcgagtttatggccgttcttggaccatcgggaagtggcaaatctacgctactaaacgcagtcgcagggagactccatggatcaaacctcaccggaaaaatacttataaacgacggtaaaataacgaaacaaacattaaaacgcaccggatttgtagcacaagacgatcttctttaccctcacttaaccgtacgtgaaaccttagttttcgtggctttgcttcgtcttcctcggagtctaacaagagacgttaaaatcagagccgctgaatcggttatatcggagctaggtttaacgaaatgtgagaacacagtcgttgggaacactttcattagaggtatctctggtggagaaaggaaacgagttagtatagctcatgaattgcttataaacccgagtcttttggttcttgatgagcctacttcgggtcttgatgctacggcggcgctccggttggttcagactcttgccgggttggctcatgggaaaggaaaaacggtggttacgtcgattcatcagccgtcgagtcgtgtgtttcagatgtttgatacggtgcttcttttgagtgaaggaaagtgtttgttcgtcggaaaaggaagagacgccatggcttactttgaatccgtcggattttcgccggcttttcctatgaatccggctgattttcttcttgatcttgctaacggagtttgtcagactgacggtgtaacggaacgggaaaagccaaacgtgagacaaacgctggtcacggcttatgatacattgctagccccacaagtcaaaacttgtatcgaagtgtcacattttcctcaagacaacgcgcgtttcgtgaaaacgcgagtaaacggcggtggaataacaacatgtattgcaacatggtttagccaactctgcattctcctccacagacttttaaaagaacggcgtcacgaatcctttgacttacttcgcattttccaagtcgtcgcggcttcgatcctttgtggtctcatgtggtggcactctgattatcgagacgtacatgaccgactaggcctactcttcttcatatccatcttttggggagtacttccatcatttaacgcggtcttcacgtttccgcaagaacgtgcaattttcactagagagcgtgcgtccggtatgtacacactctcttcttacttcatggcccatgtactcggatcgctctccatggagctcgttcttccggcatcattcttgacgttcacttattggatggtctatctacgccccgggatagtcccattcctcctcaccctctccgtgcttttactatacgttttagcgtcacaaggacttggacttgcccttggtgcagcgatcatggacgctaaaaaagcgtctacaatcgtgaccgtgacaatgctagcgtttgtcttaaccggtggttactacgtcaacaaagtgccatccggaatggtgtggatgaaatacgtttccacgacgttttattgttaccgtcttttagtggcgatccaatacgggagtggggaagagatattgcgaatgcttggatgtgattcaaagggtaagcaaggagcgagtgcggcgacatcggctggatgtcgatttgtggaggaggaagtgatcggagatgttgggatgtggacgagcgttggtgttttgtttttgatgttttttgggtatagagtattggcatatctagctttgagacgtatcaaacattaaataaagagagggagagggggagaaagttaaataaaaatgagtgggtaaatttaatttttagggttctagttcggaatattaaaacgtcgctaattgtaattttgcgatttagcgacgggttcagtttttaacatgtgaaaatgaaaatgacaagtatttttaattgtttttaataaaatgtatattttcacctaaaaaaaaaaaaaaaa

SEQ ID NO: 2 - protein, 662 aa, Arabidopsis thaliana
MSAFDGVENQMNGPDSSPRLSQDPREPRSLLSSSCFPITLKFVDVCYRVKIHGMSNDSCNIKKLLGLKQKPSDETRSTEERTILSGVTGMISPGEFMAVLGPSGSGKSTLLNAVAGRLHGSNLTGKILINDGKITKQTLKRTGFVAQDDLLYPHLTVRETLVFVALLRLPRSLTRDVKIRAAESVISELGLTKCENTVVGNTFIRGISGGERKRVSIAHELLINPSLLVLDEPTSGLDATAALRLVQTLAGLAHGKGKTVVTSIHQPSSRVFQMFDTVLLLSEGKCLFVGKGRDAMAYFESVGFSPAFPMNPADFLLDLANGVCQTDGVTEREKPNVRQTLVTAYDTLLAPQVKTCIEVSHFPQDNARFVKTRVNGGGITTCIATWFSQLCILLHRLLKERRHESFDLLRIFQVVAASILCGLMWWHSDYRDVHDRLGLLFFISIFWGVLPSFNAVFTFPQERAIFTRERASGMYTLSSYFMAHVLGSLSMELVLPASFLTFTYWMVYLRPGIVPFLLTLSVLLLYVLASQGLGLALGAAIMDAKKASTIVTVTMLAFVLTGGYYVNKVPSGMVWMKYVSTTFYCYRLLVAIQYGSGEEILRMLGCDSKGKQGASAATSAGCRFVEEEVIGDVGMWTSVGVLFLMFFGYRVLAYLALRRIKH

SEQ ID NO: 3 - DNA, 22 bp, Artificial
  primer
tgtaatgggtaatgcgataaaa

SEQ ID NO: 4 - DNA, 22 bp, Artificial
  primer
atctttggtattgaaaccatgc

SEQ ID NO: 5 - DNA, 21 bp, Artificial
  primer
tacctcgggttcgaaatcgat

SEQ ID NO: 6 - DNA, 23 bp, Artificial
  primer
cacggcttatgatacattgctaa

SEQ ID NO: 7 - DNA, 20 bp, Artificial
  primer
gagtgtgtacataccggacg

SEQ ID NO: 8 - DNA, 22 bp, Artificial
  primer
tttggttcttgatgagcctact

SEQ ID NO: 9 - DNA, 22 bp, Artificial
  primer
aagtactccccaaaagatggat

SEQ ID NO: 10 - DNA, 20 bp, Artificial
  primer
gacctgcctcatcatactcg

SEQ ID NO: 11 - DNA, 22 bp, Artificial
  primer
ttcctcaatctcatcttcttcc

SEQ ID NO: 12 - DNA, 30 bp, Artificial
  primer
caccatccatatttttatcctgatcgtgtt

SEQ ID NO: 13 - DNA, 26 bp, Artificial
  primer
aaagctgacattagtgttcctttgta

SEQ ID NO: 14 - DNA, 22 bp, Artificial
  primer
caccatgtcagcttttgacggc

SEQ ID NO: 15 - DNA, 25 bp, Artificial
  primer
cctctccctctctttatttaatgtt

SEQ ID NO: 16 - protein, 14 aa, Artificial
  peptide
QKPSDETRSTEERT

SEQ ID NO: 17 - protein, 12 aa, Artificial
  peptide
GKITKQTLKRTG

SEQ ID NO: 18 - protein, 13 aa, Artificial
  peptide
GVTEREKPNVRQT

SEQ ID NO: 19 - DNA, 1839 bp, Oryza sativa
  CDS (1)..(1839)
atggactgcttcttgtcctccgtctgcacgccgctcaatctccagttcatcgacgtggcgtaccgggtcaaggtgtcgaccacggcggcggcggcgaagggggcgccgccggggaggatatcgcacgccgggggaacaggtggtggcggtggcgcgcaggaggagaggacgatactgaaggggatcaccggcgaggcgaggccgggggaggtgctggcggtgctgggcccatcggggagcggcaagtcgacgctgctctccatcctcggcggccgcctcgccggccgccacgccgggacggtgctggcgggcgggcgcgcgccgtgccgcgccgtgcagcggcgcacggggttcgtggcgcaggacgacgtgctccacccccacctcaccgtccgcgagacgctcctcttctgcgccatgctccgcctcccggcctcctcccccgcagccgccaaggccgccgccgccgaggccgtgatcgccgagctggggctcgcgccgtgcgccgacaccatcgtcggcaacgcgttcgtccgcggcgtgtcggggggcgagcggaagcgcgtcagcatcgggcacgagctgcttgtgaacccgagcctcctcgtcctcgacgagcccacctccggcctcgactccaccgccgccgcgcgcctcgtcgccacgctctcgtcgctggcgcgcaggggccgcaccgtcgtcatgtccgtccaccagccgtccacccgcgtctaccgcatgttcgactccgtcctcctcctcgccgaggggacctgcctctacttcggcgccggccgcgacgccatggactacttcgccgccgtcggcttctcgccggccttccacgtcaaccccgccgacttcatgctcgaccttgccaacggattcgctcaaacagagtatgataattgcaacgcagccgatggcggcaacgtgaagcagtcgctgatctcgtcgtacaaccgagtgctcgcgccgcgggtgaaggcgtccatcaacgccggcgacgtccacggcggcgagcagcagccaccgccgccggcggcggagagctgcagcgggtgcacgagctggtcgaaccagttcgcgatcctgctgcggcggagcctcaaggagcggcggcacgaggcgttcacgtcgctgcggctgttccagatcatcgcgccggcgctggtggcgggggccatgtggtggcggtcgtcgccggcggcggtgggcgaccggatggggctcctcttcttcgtctccatcttctggggtgtgttcgcctccttcaacgccgtgttcgcgttcccgcaggagcggcccgtgctggcgcgggagcgcgcgtcggggatgtacgcgctctcgtcctacttcatgtcgcgcatggcgggcgacctccccatggagctcgcgctccccgccgcgttcaccgtcatcgtctacctcatggccgggctcaacccgtcgccggccgcgttcgcgctcacgctcgccgtgatcctctcctacgtgctcgtcgccgaggggctcggcctcgccgtcggcgccgtcatgatggacgccaagcgcgcctccacgctcgtcaccgtcgtcatgctcgcctacctcctcaccggcggcttctacgtccacaacgtgccggggttcatggcgtgggccaagtacacctccttcacctactactgctaccggctgctcatcgccgtgcagtacagcggccgcctcgcccgcctcctcccgccggaggaggcccgcggcgaggccagcccggccgcctgcgtcgccgcgctcgtcgccatgttcttcgcctaccggctgctcgcctacctcgcgctccgccgggtcaggaagtga

SEQ ID NO: 20 - protein, 612 aa, Oryza sativa
MDCFLSSVCTPLNLQFIDVAYRVKVSTTAAAAKGAPPGRISHAGGTGGGGGAQEERTILKGITGEARPGEVLAVLGPSGSGKSTLLSILGGRLAGRHAGTVLAGGRAPCRAVQRRTGFVAQDDVLHPHLTVRETLLFCAMLRLPASSPAAAKAAAAEAVIAELGLAPCADTIVGNAFVRGVSGGERKRVSIGHELLVNPSLLVLDEPTSGLDSTAAARLVATLSSLARRGRTVVMSVHQPSTRVYRMFDSVLLLAEGTCLYFGAGRDAMDYFAAVGFSPAFHVNPADFMLDLANGFAQTEYDNCNAADGGNVKQSLISSYNRVLAPRVKASINAGDVHGGEQQPPPPAAESCSGCTSWSNQFAILLRRSLKERRHEAFTSLRLFQIIAPALVAGAMWWRSSPAAVGDRMGLLFFVSIFWGVFASFNAVFAFPQERPVLARERASGMYALSSYFMSRMAGDLPMELALPAAFTVIVYLMAGLNPSPAAFALTLAVILSYVLVAEGLGLAVGAVMMDAKRASTLVTVVMLAYLLTGGFYVHNVPGFMAWAKYTSFTYYCYRLLIAVQYSGRLARLLPPEEARGEASPAACVAALVAMFFAYRLLAYLALRRVRK

(19) United States
(12) Patent Application Publication
(10) Pub. No.: US 2012/0272401 A1
(43) Pub. Date: Oct. 25, 2012

(54) PLANT OVEREXPRESSING ABSCISIC ACID TRANSPORTER PROTEIN AND METHOD FOR PRODUCING THE SAME

(75) Inventors: Takashi Kuromori, Kanagawa, (JP); Kazuo Shinozaki, Kanagawa (JP)

(73) Assignee: RIKEN

(21) Appl. No.: 13/518,303

(22) PCT Filed: Dec. 21, 2010
(86) PCT No.: PCT/JP2010/073664

§ 371 Date: Jun. 21, 2012

(30) Foreign Application Priority Data
Dec. 21, 2009 (JP) 2009-289457

(51) Int. Cl. A01H 5/00; A01H 5/10; A01H 1/00; C12N 5/10

(52) U.S. Cl. 800/278; 800/298; 435/419

(57) ABSTRACT

The present invention relates to a transgenic plant tolerant to environmental stress that comprises DNA encoding an exogenous abscisic acid (ABA) transporter protein in an expressible manner, a progeny thereof, or a cell, tissue or seed from such plant. The present invention also relates to a method for producing such a plant.

### TECHNICAL FIELD

The present invention relates to a transgenic plant tolerant to environmental stress that comprises DNA encoding an exogenous abscisic acid (ABA) transporter protein in an (over-)expressible manner and a method for producing the same.

### BACKGROUND ART

Abscisic acid (ABA) which is a phytohormone plays a variety of key roles in plant growth or development, such as maturation of germ and seed or postgemminative growth, and in stress response so as to adapt to environmental changes (Non-Patent Document 1). Up to the present, many signal-related molecules associated with ABA signaling have been found (Non-Patent Documents 1 to 3). In the ABA signaling mechanism, the presence of a plurality of signaling pathways has been shown, and many factors directly or indirectly influence each other in such pathways (Non-Patent Documents 2 and 3). In particular, a plurality of receptors that receive ABA have been reported recently as a result of analysis of various phenomena (Non-Patent Documents 4 to 8). To comprehensively understand the regulatory mechanism of ABA, integrative study of intercellular functions of ABA is necessary, in addition to study of intracellular signaling induced by ABA receptors. Actually, the intercellular function of ABA has been predicted to exist in plants. For example, it is known that although ABA is mainly produced in vascular tissue, it acts on guard cells located distant from the tissue to regulate stomatal aperture (Non-Patent Documents 9 to 14). However, the intercellular ABA transport mechanism and the transport factor that is responsible for ABA transport are unknown.

The ATP-Binding Cassette (ABC) transporters constitute a family of proteins having ATP-binding cassettes, which are highly conserved among prokaryotes and eukaryotes (Non-Patent Document 15). The gene cluster for the half-size type in the AtABCG subfamily of the Arabidopsis ABC transporters (conventionally also referred to as the “WBC subfamily”) is the largest subfamily of the Arabidopsis ABC transporters and the subfamily is composed of 28 genes (Non-Patent Document 16). Functions of the three members of such genes have heretofore been reported, CER5/WBC12/AtABCG12 and COF1/WBC11/AtABCG 11 are necessary to transport the cuticle wax (Non-Patent Documents 17 to 22), and WBC19/AtABCG19 has been reported as serving as a factor that imparts antibiotic tolerance (Non-Patent Document 23), although functions of genes belonging to other AtABCG subfamilies are not known at all.

Patent Document 1 describes that DNA that encodes a chloroplast-localizing protein that transports ABA to the chloroplast is expressed in a plant to impart tolerance to environmental stress, such as drought stress, to the plant. Although the objective is similar, this protein differs from a protein that enables export of ABA from a cell through the cell membrane.

### PRIOR ART DOCUMENTS

Patent Document 1: JP Patent Publication (Kokai) No. 2007-222129 A
Non-Patent Document 1: Finkelstein, R. R., Gampala, S. S., Rock, C. D., 2002, Abscisic acid signaling in seeds and seedlings, Plant Cell 14: S15-S45
Non-Patent Document 2: Hirayama, T., Shinozaki, K., 2007, Perception and transduction of abscisic acid signals: keys to the function of the versatile plant hormone ABA, Trends Plant Sci. 12: 343-351
Non-Patent Document 3: Wasilewska, A. et al., 2008, An update on abscisic acid signaling in plants and more, Mol. Plant. 1: 198-217
Non-Patent Document 4: Shen, Y. Y., et al., 2006, The Mg-chelatase H subunit is an abscisic acid receptor, Nature 443: 823-826
Non-Patent Document 5: Liu, X., et al., 2007, A G protein-coupled receptor is a plasma membrane receptor for the plant hormone abscisic acid, Science 315: 1712-1716
Non-Patent Document 6: Pandey, S., Nelson, D. C., Assmann, S. M., 2009, Two novel GPCR-type G proteins are abscisic acid receptors in Arabidopsis, Cell 136: 136-148
Non-Patent Document 7: Ma, Y. et al., 2009, Regulators of PP2C phosphatase activity function as abscisic acid sensors. Science 324: 1064-1068
Non-Patent Document 8: Park, S. Y. et al., 2009, Abscisic acid inhibits type 2C protein phosphatases via the PYR/PYL family of START proteins, Science 324: 1068-1071
Non-Patent Document 9: Cheng, W. H. et al., 2002, A unique short-chain dehydrogenase/reductase in Arabidopsis glucose signaling and abscisic acid biosynthesis and functions, Plant Cell 14: 2723-2743
Non-Patent Document 10: Koiwai, N. et al. 2004, Tissue-specific localization of an abscisic acid biosynthetic enzyme, AAO3, in Arabidopsis, Plant Physiol. 134: 1697-1707
Non-Patent Document 11: Endo, A. et al., 2008, Drought induction of Arabidopsis 9-cis-epoxycarotenoid dioxygenase occurs in vascular parenchyma cells, Plant Physiol. 147: 1984-1993
Non-Patent Document 12: Christmann, A., Weiler, E. W., Steudle, E., Grill, E., 2007, A hydraulic signal in root-to-shoot signalling of water shortage, Plant J. 52: 167-174
Non-Patent Document 13: Schachtman, D. P., Goodger, J. Q. D., 2008, Chemical root to shoot signaling under drought, Trends Plant Sci. 13: 281-287
Non-Patent Document 14: Okamoto, M. et al., 2009, High humidity induces ABA 8′-hydroxylase in stomata and vasculature to regulate local and systemic ABA responses in Arabidopsis, Plant Physiol. 149: 825-834
Non-Patent Document 15: Higgins, C. F., 1992, ABC transporters: from microorganisms to man, Annu. Rev. Cell Biol., 8: 67-113
Non-Patent Document 16: Verrier, P. J. et al., 2008, Plant ABC proteins—a unified nomenclature and updated inventory, Trends Plant Sci., 13: 151-159
Non-Patent Document 17: Pighin, J. A. et al., 2004, Plant cuticular lipid export requires an ABC transporter, Science 306: 702-704
Non-Patent Document 18: Bird, D. et al., 2007, Characterization of Arabidopsis ABCG11/WBC11, an ATP binding cassette (ABC) transporter that is required for cuticular lipid secretion, Plant J. 52: 485-498
Non-Patent Document 19: Panikashvili, D. et al., 2007, The Arabidopsis DESPERADO/AtWBC11 transporter is required for cutin and wax secretion, Plant Physiol. 145: 1345-1360
Non-Patent Document 20: Ukitsu, H. et al., 2007, Cytological and biochemical analysis of COF1, an Arabidopsis mutant of an ABC transporter gene, Plant Cell Physiol. 48: 1524-1533
Non-Patent Document 21: Luo, B., Xue, X. Y., Hu, W. L., Wang, L. J., Chen, X. Y, 2007, An ABC transporter gene of Arabidopsis thaliana, AtWBC11, is involved in cuticle development and prevention of organ fusion, Plant Cell Physiol. 48: 1790-1802
Non-Patent Document 22: Samuels, L., Kunst, L., Jetter, R., 2008, Sealing plant surfaces: cuticular wax formation by epidermal cells, Annu. Rev. Plant Biol. 59: 683-707
Non-Patent Document 23: Mentewab, A., Stewart. C. N. Jr. 2005, Overexpression of an Arabidopsis thaliana ABC transporter confers kanamycin resistance to transgenic plants, Nat. Biotechnol. 23: 1177-1180

### SUMMARY OF THE INVENTION

As described above, abscisic acid (ABA) is one of the most critical phytohormones involved in responses to the stress that is dangerous to plant life, seed maturation, and senescence. ABA is mainly produced in the vascular tissue and it induces hormone responses in various cells, such as guard cells. Such ABA responses require export of ABA from an ABA-producing cell and the intercellular ABA signaling pathway. The ABA transport mechanism through the plasma membrane remained unknown.

The present inventors aim to find a transporter that is responsible for ABA transport and ABA responses using a plant of the family Brassicaceae (Arabidopsis) as an example.

The present inventors isolated AtABCG25, which is one of ATP-binding cassette (ABC) transporter genes of Arabidopsis, by screening for ABA-sensitive mutants. AtABCG25 is expressed mainly in vascular tissue. The AtABCG25 protein fused with a fluorescent protein was localized to the plasma membrane in plant cells. It was demonstrated that the AtABCG25 protein transports ABA in an ATP-dependent manner using membrane vesicles extracted from insect cells expressing AtABCG25. It was shown that the plants overexpressing AtABCG25 have high leaf temperature and stomatal regulation was influenced therein. These results strongly suggest that the AtABCG25 protein is an ABA transporter and it is involved in the intercellular ABA signaling pathway. The existence of the ABA transport mechanism reveals the existence of active control of ABA responses to environmental stress between plant tissues or in the entire plant.

As used herein, the AtABCG25 protein from Arabidopsis thaliana and homolog (including ortholog) proteins from other plants having functions equivalent to the AtABCG25 protein are collectively referred to as “abscisic acid (ABA) transporter proteins.”

The finding obtained for Arabidopsis thaliana in the present invention is applicable to any plants having the ABA transport mechanism as a general phenomenon.

Accordingly, the present invention is summarized as follows.

(1) A transgenic plant tolerant to environmental stress, which comprises DNA encoding an exogenous abscisic acid (ABA) transporter protein in an expressible manner, wherein the ABA transporter protein is a protein having biological activity of exporting ABA from a cell through a cell membrane.

(2) The transgenic plant according to (1), wherein the DNA encoding the ABA transporter protein is any of polynucleotides (DNAs) (a) to (d) below:

(a) DNA comprising a nucleotide sequence encoding a protein comprising the amino acid sequence from Arabidopsis thaliana as shown in SEQ ID NO: 2 or the amino acid sequence from rice as shown in SEQ ID NO: 20;

(b) DNA comprising a nucleotide sequence encoding an amino acid sequence of a homolog of the protein as recited in (a), which is derived from a plant other than the plant as recited in (a) and has ABA transport activity;

(c) DNA comprising a nucleotide sequence encoding an amino acid sequence having 70% or higher identity with the amino acid sequence as shown in SEQ ID NO: 2 or 20 or an amino acid sequence of the homolog and having ABA transport activity; and

(d) DNA comprising a nucleotide sequence encoding an amino acid sequence having substitution, deletion, or addition of one or a plurality of (and preferably 1 or several) amino acids in the amino acid sequence as shown in SEQ ID NO: 2 or 20 or an amino acid sequence of the homolog and having ABA transport activity.

(3) The transgenic plant according to (2), wherein DNA encoding a protein comprising the amino acid sequence as shown in SEQ ID NO: 2 or 20 comprises an ABA transporter protein-encoding sequence as shown in SEQ ID NO: 1 or 19, respectively.

(4) The transgenic plant according to any of (1) to (3), wherein the environmental stress tolerance is drought stress tolerance.

(5) The transgenic plant according to any of (1) to (4), wherein the plant is a dicotyledonous or monocotyledonous plant.

(6) A progeny of the transgenic plant defined by any of (1) to (5), which has environmental stress tolerance.

(7) A cell, tissue, or seed of the transgenic plant defined by any of (1) to (5) or the progeny defined by (6).

(8) A method for producing a transgenic plant tolerant to environmental stress that comprises DNA comprising a nucleotide sequence encoding an exogenous abscisic acid (ABA) transporter protein in an expressible manner, comprising the steps of:

introducing the DNA into a plant cell or callus so that the DNA can be expressed therein; and

regenerating a plant body from the plant cell or callus,

wherein the ABA transporter protein has biological activity of exporting ABA from a cell through a cell membrane.

(9) A method for imparting tolerance to environmental stress to a plant comprising the steps of:

introducing into a plant or its cell DNA comprising a nucleotide sequence encoding an exogenous ABA transporter protein so that the plant or the cell comprises the DNA in an expressible manner; and

thereby imparting tolerance to environmental stress to the plant,

wherein the ABA transporter protein has biological activity of exporting ABA from a cell through a cell membrane.

(10) The method according to (8) or (9), wherein the DNA is as defined in (2) or (3).

The present invention reveals a transporter involved in the ABA transport mechanisms of plants, and provides remarkable effects that plants in which DNA comprising a nucleotide sequence encoding such a transporter (i.e., the ABA transporter protein) is overexpressed have tolerance to environmental stress, such as drought stress.

The contents as disclosed in the description and/or drawings of Japanese Patent Application No. 2009-289457, to which the present application claims priority, are incorporated herein by reference.

### BRIEF DESCRIPTION OF THE DRAWINGS

FIG. 1 shows identification of the AtABCG25 gene and the atabcg25 mutant alleles. (A) shows isolation of ABA-sensitive mutants by 96-well multititer plate assays. Mutants (atabcg25-1 and atabcg25-2) are more sensitive to a 1.0 μM ABA solution than wild-type plants (Nos and Ler). This titer plate was incubated in a growth chamber under long-day conditions for 7 days. (B) shows the structure of the AtABCG25 gene and insertional mutation sites of two atabcg25 alleles. Square boxes represent exons and black bars represent introns. Triangles represent transposon insertions in atabcg25-1 and atabcg25-2. (C) shows AtABCG25 transcripts in wild-type plants and mutants analyzed by RT-PCR. RNAs were prepared from wild-type plants (WT) and two atabcg25 mutants (atabcg25) (i.e., Nossen (Nos), Landsberg (Ler), atabcg25-1 (−1), and atabcg25-2 (−2)). Acting (ACT2) was used as a constitutively-expressed gene control. (D) to (F) show ABA-sensitive phenotype of atabcg25-1. For wild-type plant (WT) and atabcg25-1 mutant (25-1), numbers of individuals resulted in seed germination (D) and postgerminative growth (E) with ABA at several different concentrations were counted on day 2 (D) and day 4 (E). The value represents mean±s.d. for cases where 50 seeds were used (obtained from 3 independent experiments). Seedlings of wild-type plant (WT) (F, left) and atabcg25-1 (atabcg25-1) (F, right) germinated in the presence of 1.0 μM of ABA were photographed. Fifty seeds were sown and allowed to grow on a plate for 18 days in each case.

FIG. 2 shows expression patterns of the AtABCG25 gene in plant organs. (A) shows the expression pattern of AtABCG25 in plant organs by RT-PCR analysis. RNAs were prepared from seedling (Se), root (R), leaf (L), stem (S), flower (F), and fruit (Fr) of a wild-type plant. ACT2 was used as a control. (B) to (G) show results of GUS staining of 12-day-old plants (B to D) and 5-week-old leaves (E to G) without ABA treatment (B and E), after treatment with water (C and F), or after treatment with 10 μM ABA (D and G). The scale bars in (B) to (G) indicate 2 mm.

FIG. 3 shows subcellular localization of the AtABCG25 protein. (A) and (B) show the results of transient expression in the onion epidermis. Yellow fluorescent signals were observed with the YFP-AtABCG25 fusion protein (A) and YFP alone (B). (C) and (D) show subcellular localization in transgenic Arabidopsis plant. Yellow fluorescent signals emitted from the YFP-AtABCG25 fusion protein were observed in root tip cells (C) and in root tip cells after plasmolysis with 20% (w/v) sucrose for 10 minutes (D). A merged image of a fluorescence image (left) and a bright-field image (center) is shown on the right. The scale bars indicate 50 μm.

FIG. 4 shows uptake of radioisotope-labeled ABA by the AtABCG25 gene product. (A) shows the expression of AtABCG25 protein in Sf9 cells. The Sf9 membrane expressing AtABCG25 and the Sf9 membrane not expressing the same (10 μg/lane each) were subjected to Western blotting. The arrow corresponds to the AtABCG25 protein. (B) shows ATP-dependent transport of ABA by the membrane vesicle expressing AtABCG25 in the presence (black circle) or absence (white circle) of ATP. (C) shows dose dependence of ABA uptake. ATP-dependent ABA uptake was measured for 15 seconds at the indicated ABA concentration. The inset shows Lineweaver-Burk plot. (D) shows energy dependence of ABA uptake. Assay was carried out in the presence of 4 mM of the indicated nucleotide. Several experiments were carried out in the presence of 4 mM of the indicated nucleotide or 1 mM vanadate, in addition to ATP. ABA uptake in the absence of ATP is also shown (No ATP). (E) shows C is inhibition of ABA uptake. ABA uptake in the presence of ATP and a compound at the indicated concentration was measured. Full activity (100%) corresponds to 8.3 μmol/mg protein at 15 seconds (gray bar). Each value represents mean±s.d. of 3 measurements. GA represents gibberellic acid, IAA represents indoleacetic acid, JA represents jasmonic acid, PAH represents p-aminohippurate, SA represents salicylic acid, and TEA represents tetraethylammonium.

FIG. 5 shows characterization of plants overexpressing AtABCG25. (A) shows RT-PCR analysis of the expression of AtABCG25 in the plants overexpressing AtABCG25. RNAs were prepared from control plants (Cont-1 and Cont-2) and three 35S::AtABCG25 transgenic lines (OE-04, OE-14, and OE-41). ACT2 was used as a control. (B) and (C) show ABA sensitivity of postgerminative growth of the plants overexpressing AtABCG25. Seedlings of control plants (Cont-1 and Cont-2) and seedlings of three transgenic lines (OE-04, OE-14, and OE-41) expressing the 35S::AtABCG25 transgene were allowed to grow for 7 days in the presence of ABA at different concentrations (B). The value represents mean±s.d. for 50 seeds (obtained from 3 independent experiments). The seedlings germinated in the presence of 1.0 μM ABA were photographed. Fifty seeds were sown in each case and allowed to grow on a plate for 15 days (C). (D) shows thermographic images of the plants overexpressing AtABCG25. Images of 4-week-old control plants (Cont-1-1 and Cont-1-2) and 4-week-old plants overexpressing AtABCG25 (OE-04-1, OE-04-2, OE-14-1, OE-14-2, OE-41-1, and OE-41-2) were obtained using infrared thermography device (atmospheric temperature: 22° C.±2° C.; relative humidity: 60% to 70%).

FIG. 6 shows atabcg25-3 and atabcg25-4 mutant alleles and phenotypes thereof. (A) shows the insertional mutation sites of two additional atabcg25 alleles. T-DNA insertions in atabcg25-3 (SALK_{—}098823) and atabcg25-4 (SALK_{—}128331) are indicated by black triangles. (B) shows AtABCG25 transcripts in wild-type plant, and atabcg25-3 and atabcg25-4 mutants analyzed by RT-PCR. RNAs were prepared from seedlings of wild-type plant (Col) and two atabcg25 mutants (atabcg25-3 and atabcg25-4). Actin 2 (ACT2) was used as a control. (C) and (D) show ABA-sensitive phenotypes of atabcg25-3 and atabcg25-4. The number of individuals that underwent postgerminative growth in the presence of ABA at different concentrations was counted on day 11 (C). The value represents mean±s.d. for 50 seeds (obtained from 3 independent experiments). Wild-type plant and atabcg25 mutants germinated in the presence of 0.5 μM ABA were photographed (D). Fifty seeds were sown in each case and allowed to grow on a plate for 16 days.

FIG. 7 shows GUS staining of the enhancer-trap line atabcg25-2. The atabcg25-2 (CSHL_ET7134) mutant has a Ds insertion element comprising GUS reporter gene for detecting expression under the control of the original promoter or enhancer from AtABCG25. Two-week-old plants were used for GUS staining in (A). (B) shows an enlarged diagram of the roots of the 3-week-old plants being stained. (C) shows a rosette leaf of the 3-week-old plant being stained. The plant was longitudinally sectioned using the Technovit 7100 Plastic Embedding Kit (Kulzer). Xy stands for a xylem. The scale bars indicate 1 mm (A) and 50 μm (B and C).

FIG. 8 shows subcellular localization of the AtABCG25 protein. It shows transient expression in the onion epidermis. Yellow fluorescent signals are emitted from the YFP-AtABCG25 fusion protein. A merged image of a fluorescence image (left) and a bright-field image (center) is shown on the right. The lower panel shows an enlarged diagram of the boxed region. The scale bars indicate 50 μm.

FIG. 9 shows the percentage of transpiration of the plants overexpressing AtABCG25. Six- to seven-week-old leaves of three 35S::AtABCG25 transgenic lines (OE-04, OE-14, and OE-41) and wild-type plant (Col) were used. The amount of transpiration of the plants overexpressing AtABCG25 was determined as a percentage of the initial weight of a fresh leaf. The value represents mean±s.d. for 5 leaves obtained from 3 independent plants.

FIG. 10 shows drought tolerance of plants overexpressing AtABCG25. Thermographic images of the plants overexpressing AtABCG25 before drought treatment are shown in (A). Images of 6-week-old control plants (Cont-1 and Cont-2) and 6-week-old AtABCG25-overexpressing plants (OE-04 and OE-14) were obtained using infrared thermography device. The leaf temperature of the plants overexpressing AtABCG25 is higher than that of control plants. Photographs of plants after drought treatment are shown in (B). Such plants were prepared by dehydrating (stopping the water supply to) 6-week-old plants for 14 days and allowing the plants to reabsorb water for 5 days.

FIG. 11 shows the phylogenetic tree of the amino acid sequences of AtABCG9 (WBC9), AtABCG14 (WBC14), AtABCG21 (WBC21), AtABCG22 (WBC23), AtABCG25 (WBC26), AtABCG26 (WBC27), AtABCG27 (WBC28), and Os11g07600 proteins belonging to the AtABCG subfamily. Alignment of amino acid sequences was performed using Genctyx (Genetyx Corporation), which is software for processing genetic information, and the command Multiple Sequence Analysis.

FIG. 12 shows alignment of amino acid sequences of AtABCG25 (Arabidopsis thaliana, upper tier) and Os11g07600 (rice, lower tier) proteins. Boxes in the figure indicate common (or identical) amino acid residues between two sequences.

FIG. 13 shows a chart showing the stomatal apertures (μm) of the rosette leaves of the 35S::AtABCG25 transgenic plant line (OE-41) and the control plant (Col.) (4-week-old each) measured using Suzuki's universal method of printing (SUMP). N represents the number of samples. The results shown in the figure indicate that the stomatal aperture in the mature leaves of the plants overexpressing AtABCG25 is smaller than that in control plant.

FIG. 14 demonstrates that stomatal aperture of plants overexpressing AtABCG25 (OE) changes depending on CO₂ concentration and light/dark conditions, as with the case of wild-type plants (WT). (A) shows stomatal conductance (mol H₂O/m² s) of rosette leaves of 5-week-old plants determined using portable photosynthesis measurement equipment (LI-6400, LI-COR Biosciences). CO₂ concentration was regulated at intervals of 30 minutes as shown in the figure. (B) shows stomatal conductance measured during the course of light (day) for 2 hours, dark (night) for 8 hours, and light (day) for 2 hours as indicated.

FIG. 15 shows data that genetically verify that AtABCG25 is associated with the abscisic acid (ABA) signaling pathway. (A) shows pot locations, (C) shows plants, (B) shows expression of AtABCG25, NCED3, and ACT2 (control) genes analyzed by RT-PCR, and (D) shows thermographic image of the plants obtained using an infrared camera (Neo Thermo TVS-700) for plants overexpressing AtABCG25 (OE), wild-type plants (WT), mutant plants deficient in nced3 (nced3-2), and hybrids of the plant overexpressing AtABCG25 and mutant plant deficient in nced3 (nced3-2/OE) (all plants are 5 weeks old). NCED stands for “9-cis-epoxycarotenoid dioxygenase.” NCED3 is a key gene for ABA synthesis (i.e., the gene for an enzyme that catalyzes the biosynthesis of xanthoxin from 9-cis-violaxanthin). Since the NCED3-deficient mutant (nced3-2) has difficulty in closing its stoma, leaf temperature is not raised (FIG. 15D). Leaf temperature is not raised in the hybrid (nced3-2/OE) of such deficient mutant (nced3-2) and AtABCG25-overexpressing plant (OE) (FIG. 15D). It is thus verified that AtABCG25 is located downstream of NCED3 in the ABA signaling pathway.

### EMBODIMENTS OF THE INVENTION

The first aspect of the present invention provides a transgenic plant tolerant to environmental stress which comprises DNA encoding an exogenous abscisic acid (ABA) transporter protein in an expressible manner and a method for producing the same.

As described in the Background Art section, ABA which is a phytohormone plays a variety of key roles in plant growth or development, such as maturation of germ and seed or postgerminative growth, and stress response so as to adapt to environmental changes (Finkelstein, R. R., Gampala, S. S., Rock, C. D., 2002, Plant Cell 14: S15-S45). The new finding by the present inventors is the demonstration of the presence, and identification, of a protein factor that is directly associated with ABA transport in a plant in the genes of the ABCG subfamily among the numerous ABC transporter genes. While such finding was obtained using Arabidopsis thaliana which belongs to the family Brassicaceae (Arabidopsis) as a plant, the present invention should be applicable to all plants having the ABA transport mechanism. Examples of such plants include dicotyledonous and monocotyledonous plants.

The term “abscisic acid (ABA) transport mechanism” used herein refers to a mechanism in which ABA in a plant cell is exported from the cell through a cell membrane by the ABA transporter protein, and the exported ABA is involved in the intercellular ABA signaling pathway. Accordingly, the chloroplast-localizing protein described in JP Patent Publication (Kokai) No. 2007-222129 A is not the ABA transporter protein according to the present invention.

The term “abscisic acid (ABA) transporter protein” used herein refers to a protein having a function (or action) of exporting ABA in a plant cell from the cell through a cell membrane.

According to the present invention, tolerance to environmental stress, and preferably tolerance to drought stress, can be imparted to a plant when DNA encoding the ABA transporter protein is expressed (or overexpressed) therein. Examples of environmental stress include salt stress, low-temperature stress, and osmotic stress, in addition to drought stress. Any of such stresses is regulated by ABA responses mediated by the ABA transport mechanism in a plant.

The ABA transporter protein used in the present invention can be derived from any plant, and can be any protein having ABA transport activity. The term “ABA transport activity” used herein refers to biological activity of exporting ABA, which is produced in a plant cell, from the cell through a cell membrane. Such activity is measured using the vesicle transport assay method described in the Examples below. Briefly, DNA encoding a candidate of ABA transporter protein is integrated into a baculovirus expression vector, the resulting vector is introduced into an Sf9 insect cell, and the cell membrane is then separated. The candidate of ABA transporter protein is expressed in such cell membrane. The membrane comprises inside-out membrane vesicles in which the inside and the outside are inverted. After ABA labeled with a radioisotope is incorporated into the vesicle, filtration and washing are carried out using a rapid filtration technique, the radioactivity absorbed on the filter is measured, and the export activity is determined as the amount of uptake.

Examples of the ABA transporter protein includes a protein having the amino acid sequence as shown in SEQ ID NO: 2 from Arabidopsis thaliana, a homolog thereof from another plant (including an “ortholog” herein), and a mutant of the aforementioned protein or homolog thereof having ABA transport activity. Although such a mutant may contain substitution, deletion, or addition (or insertion) of one or a plurality of amino acids in the amino acid sequence of the original protein (i.e., the protein before mutation), it should retain ABA transport activity. Such mutant can be prepared using genetic engineering techniques, such as site-directed mutagenesis or mutagenesis utilizing PCR. Genetic engineering techniques are specifically described in, for example, Sambrook, et al., Molecular Cloning: A Laboratory Manual, 1989, Cold Spring Harbor Laboratory Press, and Ausubel, et al., Current Protocols in Molecular Biology, 1994, John Wiley & Sons. Such techniques can be employed to prepare the mutant as described above.

To actually overexpress ABA transporter proteins in a plant, it is necessary to introduce DNA encoding the protein, the homolog, or the mutant into a plant cell in an expressible manner. Any known technique for transformation of plant cell can be used for introduction of DNA into a cell. Examples of such techniques include the Agrobacterium method, the particle bombardment (gene gun) method, the virus vector method, the floral dip method, the leaf disc method, the protoplast method, and the electroporation method.

According to an embodiment of the present invention, DNA encoding the ABA transporter protein is selected from the group consisting of: DNA comprising a nucleotide sequence encoding the amino acid sequence as shown in SEQ ID NO: 2 from Arabidopsis thaliana or the amino acid sequence as shown in SEQ ID NO: 20 from rice; DNA comprising a nucleotide sequence encoding an amino acid sequence of a homolog thereof from a plant other than the above having ABA transport activity: DNA comprising a nucleotide sequence encoding an amino acid sequence having 30% or higher, 40% or higher, 50% or higher, 60% or higher, 70% or higher, preferably 80% or higher, more preferably 90% or higher, and further preferably 95% or higher, 97% or higher, or 99% or higher identity with the amino acid sequence as shown in SEQ ID NO: 2 or SEQ ID NO: 20 or an amino acid sequence of the homolog and having ABA transport activity; and DNA comprising a nucleotide sequence encoding an amino acid sequence having substitution, deletion, or addition of one or a plurality of, and preferably one or several, amino acids in the amino acid sequence as shown in SEQ ID NO: 2 or SEQ ID NO: 20 or an amino acid sequence of the homolog and having ABA transport activity.

Conservative amino acid substitution is preferable in the present invention. Conservative amino acid substitution refers to, for example, substitution between amino acids having similar properties in terms of structural, electrical, polar, or hydrophobic properties or the like. Such properties can be classified based on, for example, similarity in amino acid side chains. Examples of amino acids having basic side chains include lysine, arginine, and histidine. Examples of amino acids having acidic side chains include aspartic acid and glutamic acid. Examples of amino acids having uncharged polar side chains include glycine, asparagine, glutamine, serine, threonine, tyrosine, and cysteine. Examples of amino acids having hydrophobic side chains include alanine, valine, leucine, isoleucine, proline, phenylalanine, and methionine. Examples of amino acids having branched side chains include threonine, valine, and isoleucine. Examples of amino acids having aromatic side chains include tyrosine, tryptophan, phenylalanine, and histidine.

An example of DNA comprising a nucleotide sequence encoding the amino acid sequence as shown in SEQ ID NO: 2 (Arabidopsis thaliana) or SEQ ID NO: 20 (rice) is DNA comprising a sequence encoding the ABA transporter protein as shown in SEQ ID NO: 1 (Arabidopsis thaliana) or SEQ ID NO: 19 (rice).

The nucleotide sequences of the DNA from Arabidopsis thaliana are registered with GenBank (NCBI, U.S.A.) under the gene identification number Atlg71960 and the accession numbers AY050810 (cDNA) and AAK92745 (protein). While a protein encoded by such DNA is described as a putative ABC transporter protein therein, it was not known at the time of registration that such protein has a function as an ABA transporter.

In addition, DNA that is hybridizable under stringent conditions to a sequence complementary to the nucleotide sequence of DNA comprising a sequence encoding the ABA transporter protein as shown in SEQ ID NO: 1 or SEQ ID NO: 19 and encodes a protein having ABA transport activity can also be used in the present invention. Such homologous DNA includes one having, for example, about 40% or higher, about 50% or higher, about 60% or higher, about 70% or higher, about 80% or higher, about 90% or higher, about 95% or higher, about 97% or higher, or about 99% or higher identity with the nucleotide sequence as shown in SEQ ID NO: 1 or SEQ ID NO: 19 and encoding a protein having ABA transport activity. DNA encoding a homolog of the ABA transporter protein derived from Arabidopsis thaliana would be within the scope of such DNA.

The term “stringent conditions” includes, for example, the condition of hybridization carried out at about 42° C. to 55° C. in the presence of 2× to 6×SSC, followed by washing once or several times at 50° C. to 65° C. in the presence of 0.1× to 1×SSC and 0.1% to 0.2% SDS. Since such conditions vary depending on the GC content of the nucleic acid as a template, ionic strength, temperature, and other factors, the conditions are not limited to those specifically described above. 1×SSC is composed of 0.15 M NaCl and 0.015 M sodium citrate (pH 7.0). In general, stringent conditions are set at a temperature lower by about 5° C. than the melting temperature (Tm) of a given sequence at the designated ion intensity and pH. Tm refers to a temperature at which 50% of the probes complementary to a template sequence hybridize to the template sequence at equilibrium.

The term “DNA” used herein refers to genomic DNA, a gene, or cDNA.

The term “identity” used herein refers to a percentage denoting the number of identical amino acids or nucleotides (or positions) relative to the total number of amino acids or nucleotides (or positions, including gaps) observed when, for example, two amino acid sequences or nucleotide sequences are aligned with or without the introduction of gaps so as to achieve the maximal match. Determination of percent identity between sequences, search of homolog sequence, or homology search can be performed by utilizing known algorithms, such as BLAST (BLASTN, BLASTP, BLASTX, etc.) or FASTA (Altschul, S. F., W., Gish, W., Miller, E. W., Myers, and D. J., Lipman, Basic local alignment search tool, J. Mol. Biol., 215 (3): 403-10, 1990).

The term “several” used herein for amino acids or nucleotides generally refers to an integer from 2 to 10, and it is preferably an integer from 2 to 5. The term “a plurality of” used herein for amino acids or nucleotides refers to an integer of 2 or greater. For example, it may be an integer from 2 to 70, 2 to 60, 2 to 50, 2 to 40, 2 to 30, 2 to 20, or 2 to 10.

The term “homolog” used herein encompasses all ABA transporter polypeptides that are derived from plants other than Arabidopsis thaliana and have ABA transport activity. Such homolog can be obtained by accessing web sites of organizations that disclose plant genomes, such as NCBI (U.S.A.), EBI (Europe), KAOS (Kazusa DNA Research Institute, Japan), IRGSP (International Rice Genome Sequencing Project, Japan), GrainGenes (U.S.A.), PGDIC (U.S.A.), ForestGEN (Forestry and Forest Products Research Institute, Japan), RAP-DB (Ministry of Agriculture, Forestry and Fisheries, Japan), and the Rice Genome Annotation Project Database (NSF, U.S.A.).

Such homologs are naturally-occurring polypeptides having ABA transport activity of plants, and they may be derived from any of dicotyledonous or monocotyledonous plants having ABA transport mechanisms. For example, a rice (Oryza sativa) homolog is identified by the gene identification number Os11g0177400 and the accession numbers NM_{—}001072418 (partial cDNA) and NP_{—}001065886 (the accession numbers of RAP-DB, Ministry of Agriculture, Forestry and Fisheries, Japan) or the gene identification number Os11g07600 (the accession numbers of the Rice Genome Annotation Project, NSF, U.S.A.), and a Lotus japonicus homolog is identified by the gene identification number LjSGA_{—}111595.1 and the accession number BABK01078073 (the genome shotgun sequence) (DNA Research, 2006, 13, 205-228).

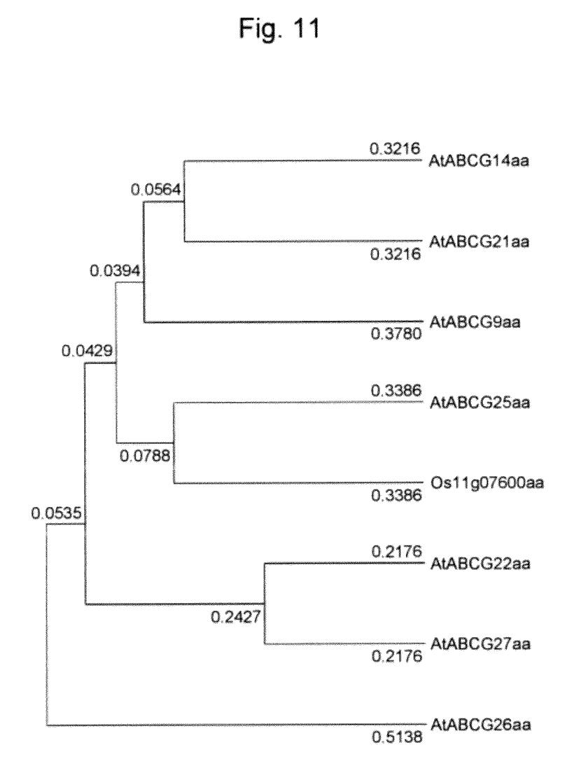

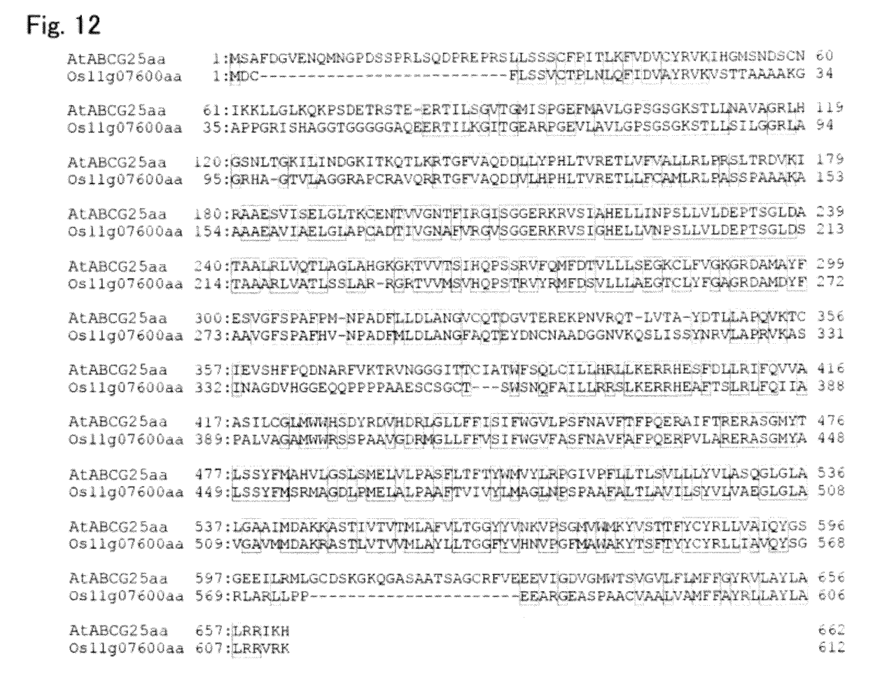

The ABA transporter protein AtABCG25 (SEQ ID NO: 2) from Arabidopsis thaliana and the ABA transporter protein Os11g07600 (SEQ ID NO: 20) from rice are very closely related to each other as seen from the phylogenetic tree of ABCG (WBC) family members (FIG. 11) and the alignment (FIG. 12).

In addition, ABA transporter proteins have common functional domains, such as the ATP-binding site and a membrane region. In the case of the amino acid sequence of AtABCG25 (WBC26) (SEQ ID NO: 2), for example, the ATP-binding site is located from amino acid position 71 (proline) to amino acid position 290 (glycine), and the membrane region is located from amino acid position 408 (leucine) to amino acid position 594 (tyrosine).

For the plant transformation, target DNA is selected from a cDNA library or genomic DNA library of plant tissues (e.g., leaves, stems, roots, petals, pollen, seeds, or calluses) and integrated into an adequate vector (e.g., a phage or plasmid vector). DNAs and vectors can be manufactured using, for example, genetic engineering techniques. Genetic engineering techniques described in, for example, Sambrook, et al., Molecular Cloning: A Laboratory Manual, 1989, Cold Spring Harbor Laboratory Press; and Ausubel, et al., Current Protocols in Molecular Biology, 1994, John Wiley & Sons can be employed.

Also, in connection with the above, homolog DNA can be obtained from the cDNA library or genomic DNA library as described above using, for example, DNA comprising the nucleotide sequence as shown in SEQ ID NO: 1 or SEQ ID NO: 19, a partial sequence thereof, or a sequence complementary thereto as a (labeled) probe or primer.

Plants to be transformed are not particularly limited. Examples thereof include, but are not limited to, dicotyledonous and monocotyledonous plants, such as plants belonging to the families Brassicaceae, Gramineae, Solanaceae, Leguminosae, and Salicaceae (listed below).

Brassicaceae: Arabidopsis thaliana, Brassica rapa, Brassica napus, Brassica oleracea var capitata, Brassica rapa var. pekinensis, Brassica rapa var. chinensis, Brassica rapa var rapa, Brassica rapa var hakabura, Brassica rapa var. lancinifolia, Brassica rapa var peruviridis, Brassica Raphanus sativus, and Wasabia japonica

Solanaceae: Nicotiana tabacum, Solanum melongena, Solaneum tuberosum, Lycopersicon lycopersicum, Capsicum annuum, and Petunia

Leguminosae: Glycine max, Pisum sativum, Vicia faba, Wisteria floribunda, Arachis. hypogaea, Lotus corniculatus var. japonicus, Phaseolus vulgaris, Vigna angularis, and Acacia

Compositae: Chrysanthemum morifolium and Helianthus annuus

Arecaceae (Palmae): Elaeis guineensis, Elaeis oleifera, Cocos nucifora, Phoenix dactylifera, and Copernicia

Anacardiaceae: Rhus succedanea, Anacardium occidentale, Toxicodendron vernicifluum, Mangifera indica, and Pistacia vera

Cucurbitaceae: Cucurbita maxima, Cucurbita moschata, Cucurbita pepo, Cucumis sativus, Trichosanthes cucumeroides, and Lagenaria siceraria var. gourda

Rosaceae: Amygdalus communis, Rosa, Fragaria. Prunus, and Malus pumila var domestica

Caryophyllaceae: Dianthus caryophyllus

Salicaceae: Populus trichocarpa, Populus nigra, and Populus tremula

Myrtaceae: Eucalyptus camaldulensis and Eucalyptus grandis

Gramineae: Zea mays, Oryza saliva, Hordeum vulgare. Triticum aestivum, Phyllostachys, Saccharum officinarum, Pennisetum pupureum, Erianthus ravenae, Miscanthus virgatum, Sorghum, and Panicum

Liliaceae: Tulipa and Lilium

Briefly, for example, DNA encoding the ABA transporter protein can be amplified by a polymerase chain reaction (PCR) using primers prepared based on a known sequence (e.g., SEQ ID NO: 1 or SEQ ID NO: 19) from cDNA library from plant tissue (preferably tissue comprising vascular bundles or veins), which can be prepared using a known technique utilizing a phage. Such DNA is purified using, for example, agarose gel or polyacrylamide gel electrophoresis, and the resultant is inserted into an adequate expression vector in a manner allowing overexpression. Known techniques as described in Ausubel et al. (1994, supra) can be used for PCR techniques regarding PCR procedures, primers and the like.

Examples of vectors include binary vectors and other vectors. A binary vector comprises two border sequences of approximately 25 by (i.e., a right border (RB) sequence and a left border (LB) sequence) from Agrobacterium T-DNA, and exogenous DNA is inserted between the border sequences. Examples of binary vectors include pBI (e.g., pBI101, pBI101.2, pBI101.3, pBI121, and pBI221; Clontech), pGA482, pGAH, and pBIG vectors. Examples of other vectors include intermediate plasmids such as pLGV23Neo, pNCAT and pMON200, as well as pH35GS which comprises the Gateway cassette (Kubo et al., 2005, Genes & Dev. 19: 1855-1860). A promoter is ligated to the 5′ end of exogenous DNA. Examples of promoters include cauliflower mosaic virus (CaMV) 35S promoter, nopaline synthase gene promoter, maize ubiquitin promoter, octopine synthase gene promoter, and rice actin promoter. Further, a terminator (e.g., a nopaline synthase gene terminator) is inserted into the 3′ end of exogenous DNA. A selection marker that is necessary for selecting a transformed cell is further inserted into a vector. Examples of selection markers include drug resistance genes, such as kanamycin resistance gene (NPTII), hygromycin resistance gene (htp), and bialaphos resistance gene (bar).

Examples of transformation techniques for introducing a vector constructed in the manner described above into a plant include the Agrobacterium method, the particle bombardment (gene gun) method, the electroporation method, the virus vector method, the floral dip method, and the leaf disc method. Plant transformation techniques and tissue culture techniques are described in, for example, Ko Shimamoto, Kiyotaka Okada (ed.), Shokubutsu Saibou Kougaku Series 15, Model Shokubutsu No Jikken Protocol, Idengakuteki Shuhou Kara Genome Kaiseki Made (Plant Cell Technology Series 15, Experimental Protocol for Model Plants, From Genetic Technique to Genome Analysis), Shujunsha, 2001.

According to a method utilizing the binary vector-Agrobacterium system, plant cells, calluses, or plant tissue segments are prepared, such materials are infected with Agrobacterium, and DNA encoding the protein of the present invention is introduced into the plant cells. Upon transformation, a phenolic compound (acetosyringon) may be added to a medium, and cells of monocotyledonous plants can be efficiently transformed in particular. Agrobacterium tumefaciens strains, such as C58, LBA4404, EHA101, EHA105, or C58C1RifR, can be used as Agrobacterium.

A medium used for transformation is a solid medium. For example, 1% to 5% of saccharides, such as maltose, sucrose, glucose, or sorbitol, and 0.2% to 1% of polysaccharide solidification agents, such as agar, agarose, Gelrite, or gellan gum, can be added to a basal medium (i.e., a plant culture medium, such as MS medium, B5 medium, DKN medium, or Linsmaier & Skoog medium). Auxins, cytokinines, antibiotics (e.g., kanamycin, hygromycin, or carbenicillin), acetosyringon, and the like can be added to a medium. The pH of a medium can be adequately selected and it is, for example, between pH5 and pH7. For example, a substance that induces transcription activation, such as a steroid hormone, can be added to the medium after transformation.

Specifically, a suspension of Agrobacterium cells is prepared, the plant calluses or tissues (e.g., laminae, roots, stem segments, or meristems) are soaked in the cell suspension, moisture is removed therefrom, and the cells are then sown on a solid medium to conduct coculture. A callus is a mass of plant cells, and it can be induced from a plant tissue segment or a mature seed using a callus induction medium. A transformed callus or tissue segment is selected with the aid of a selection marker. In case of callus, the callus can then be redifferentiated into a seedling in a redifferentiation medium. In case of plant segment, a callus may be induced from the plant segment, and redifferentiated into a seedling. Alternatively, a protoplast may be prepared from the plant segment, subjected to callus culture, and then redifferentiated into a seedling. The thus-obtained seedling is transferred to soil after rooting, and regenerated into a plant body.

When the floral dip method is used, for example, a suspension of Agrobacterium cells is prepared, flower buds of a plant host to be transformed (which had been grown to develop premature flower buds) are soaked in the cell suspension for a short period of time, and the resultant is covered to maintain humidity overnight, as described in Clough and Bent et al. (Plant J. 16, 735-743, 1998). The cover is removed on the following day, the plant is allowed to grow, and seeds are then harvested. Transformed individuals can be selected by sowing the harvested seeds on a solid medium to which an adequate selection marker, such as an antibiotic, has been added. The thus-selected individuals can be transferred to soil and grown to obtain the next-generation seeds of transformed (or transgenic) plants.

A transformed plant may be subjected to crossing with a wild-type plant or self-pollination to produce a progeny having the same novel phenotype as the transformed plant.

A transformed plant or a progeny thereof produced according to the method as described above comprises DNA encoding the ABA transporter protein in a manner allowing overexpression, and exhibits tolerance to environmental stress, such as drought stress.

The term “expressible” used herein refers to a situation in which DNA encoding the exogenous ABA transporter protein can be expressed at a higher level than a control plant containing no such DNA. The expression may be any of constitutive expression, inducible expression, and autonomous expression. It is preferable that target DNA be forced to be expressed constantly under environmental stress conditions.

The second aspect of the present invention provides, in addition to the transgenic plant or a progeny thereof as described above, a cell, tissue, or seed thereof.

The third aspect of the present invention provides a method for producing a transgenic plant tolerant to environmental stress that comprises DNA encoding an exogenous ABA transporter protein in an expressible manner, comprising the steps of introducing such DNA into a plant cell or callus so that the DNA can be expressed therein, and regenerating a plant body from such plant cell or callus. The ABA transporter protein has biological activity of exporting ABA from a cell through a cell membrane.

Techniques used for transformation in this method are as described above.

The fourth aspect of the present invention provides a method for imparting tolerance to environmental stress to a plant, comprising the steps of introducing into a plant or its cell DNA encoding an exogenous ABA transporter protein so that the plant or the cell comprises the DNA in an expressible manner, and thereby imparting tolerance to environmental stress to the plant. The ABA transporter protein has biological activity of exporting ABA from a cell through a cell membrane.

Techniques used for transformation in this method are as described above.

Examples of environmental stress include drought stress, salt stress, low-temperature stress, and osmotic stress. This is because ABA is known to function when a plant receives such environmental stress. Since a plant having tolerance to drought stress can be provided according to the present invention, in particular, the present invention enables the planting of such plant in a dry zone, such as desertified land.

The present invention is described in greater detail with reference to the following Examples. However, the Examples are provided for illustrative purpose only and the technical scope of the present invention is not limited to the Examples.

### EXAMPLES

### Materials and Methods

### Plant Materials and Growth Conditions

Plants were grown on MS medium containing 1% (w/v) sucrose and 0.8% (w/v) agar or in soil at 22° C. under a 16-hour light/8-hour dark cycle. The atabcg25-1 (15-0195-1) mutant was isolated from the Ds transposon-tagged lines of Nossen ecotype (Kuromori, T. et al., 2004, Plant J. 37: 897-905). The atabcg25-2 (CSHL_ET7134) allele is a Ds transposon-tagged line of the Landsberg ecotype, and it was obtained from the Cold Spring Harbor Laboratory (Sundaresan, V. et al., 1995, Genes Dev9: 1797-1810). Genomic DNA of Arabidopsis plants was prepared using an automatic DNA isolation system P1-50 alpha (Kurabo). PCR-based genotyping was carried out using ExTaq polymerase (Takara Bio). To determine the genotype of atabcg25-1, the primers listed below were used: 15-0195_{—}5′ (5′-TGTAATGGGTAATGCGATAAAA-3′ (SEQ ID NO: 3)); 15-0195_{—}3′ (5′-ATCTTTGGTATTGAAACCATGC-3′ (SEQ ID NO: 4)); and Ds5-3 (5′-TACCTCGGGTTCGAAATCGAT-3′ (SEQ ID NO: 5)). To determine the genotype of atabcg25-2, the primers listed below were used: ET7134_{—}3′ (5″-CACGGCTTATGATACATTGCTAA-3′ (SEQ ID NO: 6)); ET7134_{—}5′ (5′-GAGTGTGTACATACCGGACG-3′ (SEQ ID NO: 7)); and Ds5-3. The presence of a wild-type allele was detected by PCR using gene-specific primers for the sequences flanking the insertion site (i.e., 15-0195_{—}5′ and 15-0195_{—}3 ¢ or ET7134_{—}3′ and ET7134_{—}5′), and the mutant alleles were detected using a Ds border primer in combination with one of the gene-specific primers (i.e., Ds5-3 and 15-0195_{—}5′ or Ds5-3 and ET7134_{—}5′). Fifty sterilized seeds were sown on a 0.5× MS medium plate containing 1% sucrose and ABA at various concentrations for germination and greening assays. Stratification was carried out at 4° C. for 4 days, germination was scored based on hypocotyl protrusion, and postgerminative growth (greening) was scored based on fully green, expanded cotyledons. The means and standard deviations (s.d.) were determined through 3 independent experiments.

### Experiments for Studying Gene Expression and GUS Staining

RNA was extracted from Arabidopsis plants for RT-PCR using the RNeasy Plant Mini Kit (Qiagen). RT-PCR was carried out using AtABCG25_RT-PCR_{—}5′ (5′-TTTGGTTCTTGATGAGCCTACT-3′ (SEQ ID NO: 8)) and AtABCG25_RT-PCR_{—}3′ (5′-AAGTACTCCCCAAAAGATGGAT-3′ (SEQ ID NO: 9)) primers with the PrimeScript One Step RT-PCR kit (Takara Bio). The Actin2 transcript as a control was amplified using Actin2RT-F (5′-GACCTGCCTCATCATACTCG-3′ (SEQ ID NO: 10)) and Actin2RT-R (5″-TTCCTCAATCTCATCTTCTTCC-3′ (SEQ ID NO: 11)) primers. GUS staining was carried out according to the standard protocol (Sundaresan, V. et al., 1995, Genes Dev9: 1797-1810). The plants stained with GUS were observed under a SZ61 stereoscopic microscope (Olympus), and digital images were obtained using the DS-L1 CCD digital camera (Nikon). Finer images were photographed using a BX60 upright microscope (Olympus) and a VB-7010 CCD camera (Keyence). For transgenic lines to be examined for GUS expression from the AtABCG25 promoter a 2-kb AtABCG25 promoter region was prepared by amplifying the region using AtABCG25pro_Forward (5′-CACCATCCATATTTTTATCCTGATCGTGTT-3′ (SEQ ID NO: 12)) and AtABCG25pro_Reverse (5′-AAAGCTGACATTAGTGTTCCTTTGTA-3′ (SEQ ID NO: 13)) primers with KOD-plus polymerase (Toyobo), cloning the amplified product into the pENTR/D/TOPO vector (Invitrogen), and integrating the resultant into the GUS-fusion vector pBGGUS (Kubo, M. et al., 2005, Genes Dev 19: 1855-1860). Leaves of 5-week-old pAtABCG25::GUS transgenic plants were soaked in 10 μM ABA for 24 hours for ABA treatment.

### Subcellular Localization

Full-length cDNA of the AtABCG25 (Atlg71960) gene was obtained from the RIKEN BioResource Center. AtABCG25 cDNA (2006-bp) was amplified using KOD-plus polymerase with AtABCG25_Forward (5′-CACCATGTCAGCTTTTGACGGC-3′ (SEQ ID NO: 14)) and AtABCG25_Reverse (5′-CCTCTCCCTCTCTTTATTTAATGTT-3′ (SEQ ID NO: 15)) primers, and the resultant was cloned into the pENTR/D-TOPO vector. The sequence of the clone (pENTR-AtABCG25) was confirmed, and it was integrated into the YFP-fusion protein vector pH35YG (Kubo M, et al., 2005, Genes Dev19: 1855-1860) using LR clonase (Invitrogen). To examine transient expression, the inner surface of an onion (Allium cepa) was placed on MS medium and bombarded with 0.15 μg of plasmid DNA coated onto 1.5 mg of 1-μm gold particles using a helium biolistic device (PDS-1000; Bio-Rad) at a pressure of 1,350 psi (10.7 MPa) according to the manufacturer's instructions. After incubation for about 16 hours, the onion epidermis was peeled off, and yellow fluorescence was examined under an LSM 510 META confocal laser scanning microscope (Carl Zeiss). Further, the present inventors introduced a YFP-fusion protein construct vector consisting of pH35YG into Arabidopsis using an Agrobacterium-mediated transformation system. Thereafter, the roots of the transgenic plants were treated with 0.5 M mannitol for 20 minutes for plasmolysis of the cells.

Preparation of Membrane Vesicles from Sf9 Insect Cells Expressing AtABCG25 and Immunoblotting

A BaculoGold™ baculovirus expression vector system (BD PharMingen) was used to prepare the recombinant baculovirus. Sf9 insect cells (Spodoptera frugiperda) were infected with the virus and cultured in SF900-SFM medium (Invitrogen) at 27° C. for 72 hours in a shaking incubator. Cells were collected by centrifugation at 1,100×g for 10 minutes and then disrupted by nitrogen cavitation in 150 mM NaCl. 3 mM CaCl₂, 2 mM MgCl₂, 0.1 mM EGTA, and 10 mM Tris-HCl (pH 7.4). Undisrupted cells, nuclear debris, and mitochondria were pelleted by centrifugation at 2,600×g for 10 minutes. The supernatant was centrifuged at 100,000×g for 30 minutes, and the pellet was resuspended in 70 mM KCl, 7.5 mM MgCl₂, and 50 mM MOPS-Tris (pH 7.0). Membrane vesicles were stored by freezing in a deep freezer until use. Concentration of the protein was measured using the BCA protein assay kit (Pierce) with bovine serum albumin as a control. To confirm the production of the AtABCG25 proteins in the Sf9 cells by Western blot analysis, an anti-AtABCG25 antibody was obtained by immunizing a rabbit with a synthetic peptide (Operon Biotechnologies). This synthetic peptide consisted of 3 types of 12 to 14 amino acid residues from the Arabidopsis AtABCG25 protein, designed based on positions 69 to 82 (QKPSDETRSTEERT), positions 132 to 143 (GKITKQTLKRTG), and positions 328 to 340 (GVTEREKPNVRQT). Membrane proteins were solubilized using 4% SDS and subjected to 10% SDS-PAGE. Proteins were transferred to a polyvinylidene difluoride membrane and probed using a rabbit anti-AtABCG25 antibody and horseradish-peroxidase-conjugated donkey anti-rabbit IgG. Specific immunoreactive proteins were detected by exposure to an autoradiography film using a chemiluminescence detection system (ECL-plus, Amersham Biosciences).

### Vesicle Transport Assay

An experiment of membrane transport was carried out using the rapid filtration technique (Otsuka, M. et al., 2005, Proc. Natl. Acad. Sci., U.S.A., 102: 17923-17928). Briefly, 100 μl of transport medium (70 mM KCl, 7.5 mM MgCl₂, 50 mM MOPS-Tris, pH 7.0) containing 15 μg of membrane proteins, 4 mM adenosine triphosphate (ATP), and 1 μM ABA (which included 22 nM DL-cis,trans-[G-³H] abscisic acid (GE Healthcare)) was incubated at 27° C. The transport medium was passed through a 0.45-μm nitrocellulose filter (Millipore), and the filter was washed with 6 ml of ice-cooled stop buffer (70 mM KCl, 7.5 mM MgCl₂, 50 mM MOPS-Tris, pH 7.0). The radioactivity retained on the filter was determined using a liquid scintillation counter (Tri-Carb2800TRs; PerkinElmer). Membrane vesicles from Sf9 cells containing only the vector were used as the controls.

### Overexpressing Arabidopsis Plants and Thermographic Imaging

To prepare the 35S::AtABCG25 plasmid, a clone (pENTR-AtABCG25) which contains the full-length AtABCG25 cDNA was integrated into the overexpression vector pGWB2. The HindIII-XbaI site in the vector was replaced by the 35S promoter from pBE2113N (Mitsuhara, I. et al., 1996, Plant Cell Physiol., 37: 49-59). The 35S::AtABCG25 plasmid was introduced into Agrobacterium GV3101 by electroporation to generate transgenic plants by the floral dipping method. From among the T2 plants, overexpressing lines were selected by examination with RT-PCR. After self-pollination, T3 seeds were used for subsequent experiments. Thermographic images were obtained using a Neo Thermo TVS-700 infrared camera (Nippon Avionics) and then analyzed using PE Professional software (Nippon Avionics). Plants were grown on soil under well-watered conditions (22° C., 60% to 70% relative humidity, 16-hour photoperiod).

### Drought Stress Assay of Overexpressing Arabidopsis Plants

Six-week-old plants, which had been grown on soil in the same vat in a plant growth chamber, were transferred to a vat containing no water, and the plants were subjected to dehydration without water supply for 14 days. Thereafter, plants were observed 5 days after water reabsorption to determine the growth rate.

### Results and Discussion

Identification of AtABCG25 Gene and atabcg25 Mutant Alleles

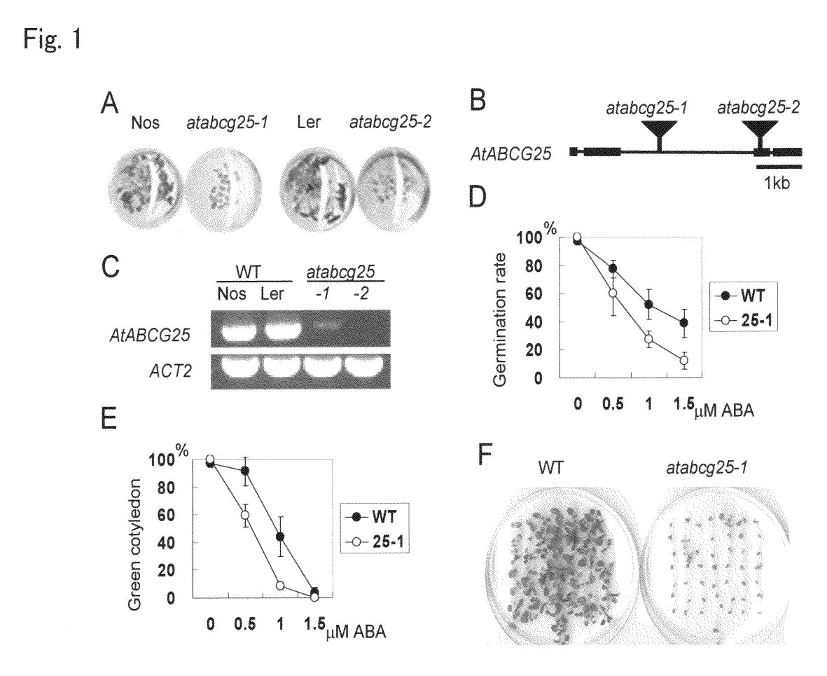

To obtain novel mutants related to ABA responses, the present inventors selected ABA-related mutants from the transposon-tagged lime collection. The present inventors previously constructed about 12,000 transposon-tagged lines of Arabidopsis using the activator (Ac)/dissociation (Ds) system and determined the sequences flanking the Ds element in all lines (Kuromori T, et al., 2004, Plant J. 37: 897-905). From them, the present inventors selected homozygous insertion lines in which the Ds transposon had been inserted into the gene-coding regions for systematic phenotyping analyses (phenome analyses) (Kuromori, T. et al., 2006, Plant J. 47: 640-651). The present inventors conducted high-throughput screening using 96-well multititer plates to screen about 2,000 homozygous insertion lines and isolated one mutant line exhibiting an ABA-sensitive phenotype at the germination and seedling stages (FIG. 1A). According to the genomic sequence flanking the Ds insertion into the isolated line (15-0195-1), the Ds element was inserted in the second intron of a gene-coding region (ORF) of the Atlg71960 gene (FIG. 1B).

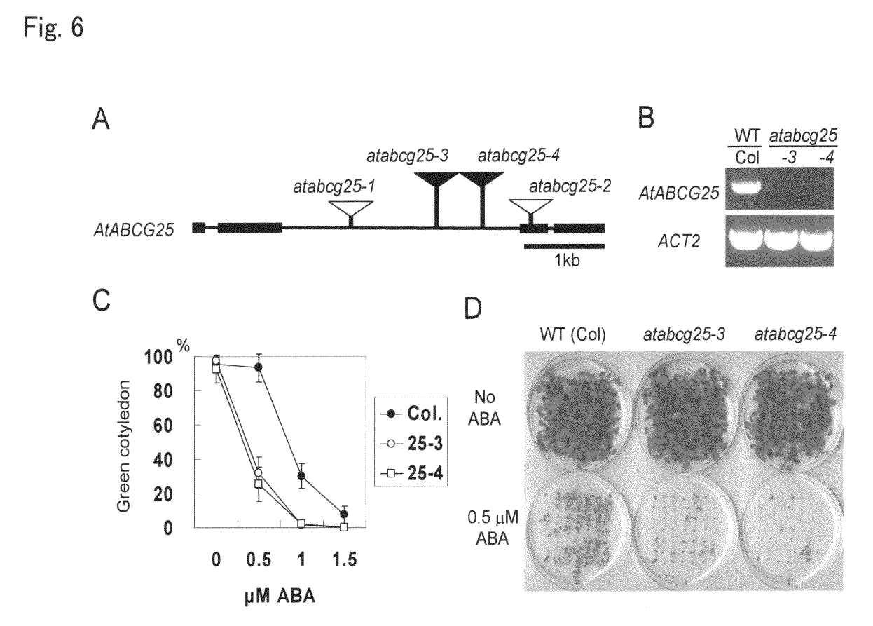

The Atlg71960 gene encodes AtABCG25 (also reported as AtWBC26), and it is a member of the ABCG subfamily of ABC (ATP-binding cassette) transporters in the Arabidopsis genome (Verrier, P. J. et al., 2008, Trends Plant Sci. 13: 151-159). The mutant obtained first was designated as atabcg25-1. The mutant line CSHL_ET7134, designated as atabcg25-2, had a Ds insertion in the third exon of AtABCG25 and exhibited the same phenotype as atabcg25-1 in the multititer plate assay (FIG. 1A). Two additional alleles from T-DNA insertion lines also exhibited ABA-sensitive phenotypes (FIG. 6). This suggests that mutation of AtABCG25 is responsible for the ABA-sensitive phenotype. PCT (RT-PCR) analysis showed that the homozygous insertional mutation line of atabcg25-2 contained no detectable amount of transcripts. This indicates that this mutant is a gene knockout mutant (FIG. 1C). While atabcg25-1 was also a knockout mutant, it resulted in a very faint band upon RT-PCR (FIG. 1C). This is probably because the insertional mutation was in a relatively long intron (FIG. 1B). All of the atabcg25 mutants exhibited ABA-sensitive phenotypes during the early growth stage (FIGS. 1D to 1F and FIG. 6).

### AtABCG25 Gene Expression Patterns in Plant Organs

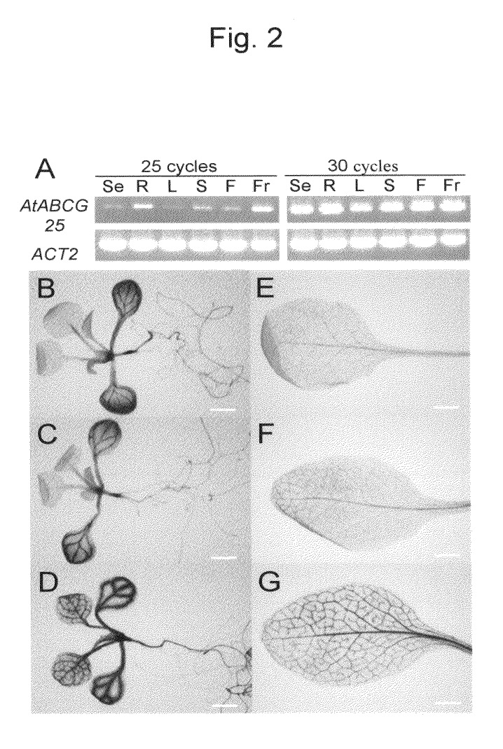

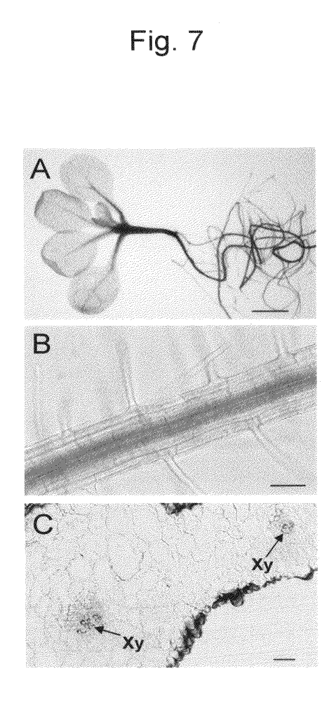

To examine the gene expression patterns of AtABCG25, RT-PCR was performed to determine the expression patterns in wild-type tissues. RNAs were extracted from seedlings, roots, stems, leaves, flowers, and fruits of wild-type plants. Transcripts for AtABCG25 were amplified from all the tissues described above (FIG. 2A). For further analyzing the tissue-specific expression, expression of GUS reporter was studied using about 2-kb AtABCG25 promoter (pAtABCG25) region. pAtABCG25::GUS transgenic plants were produced, and the GUS activities were detected mainly in the hypocotyls, roots and vascular veins of leaves in the transformants (FIGS. 2B to 2G). To check the ABA-inducibility of AtABCG25, pAtABCG25::GUS transgenic plants were treated with an ABA solution and then subjected to GUS staining. The expression levels of the GUS reporter in the transformants increased by the ABA treatment (FIGS. 2B to 2G). Additionally, the present inventors stained atabcg25-2 mutants, which contained the GUS reporter gene in the Ds element as an enhancer-trap system (Sundaresan V. et al., 1995, Genes Dev 9: 1797-1810). GUS signals in atabcg25-2 were observed in vascular tissues (FIG. 7A) and were detected along the vascular bundles in the centers of roots (FIG. 7B). When the stained leaves were cross-sectioned, the signals were accumulated in an area close to the vascular veins (FIG. 7C). Interestingly, enzymes that biosynthesize ABA are expressed in vascular parenchyma cells, and expression of the genes is increased under stress conditions in Arabidopsis (Cheng, W. H. et al., 2002, Plant Cell 14: 2723-2743; Koiwai, N et al., 2004, Plant Physiol. 134: 1697-1707; Endo, A. et al., 2008, Plant Physiol. 147: 1984-1993). These results suggest that AtABCG25 plays an important role in ABA responses at the site of its biosynthesis.

### Subcellular Localization of AtABCG25 Protein

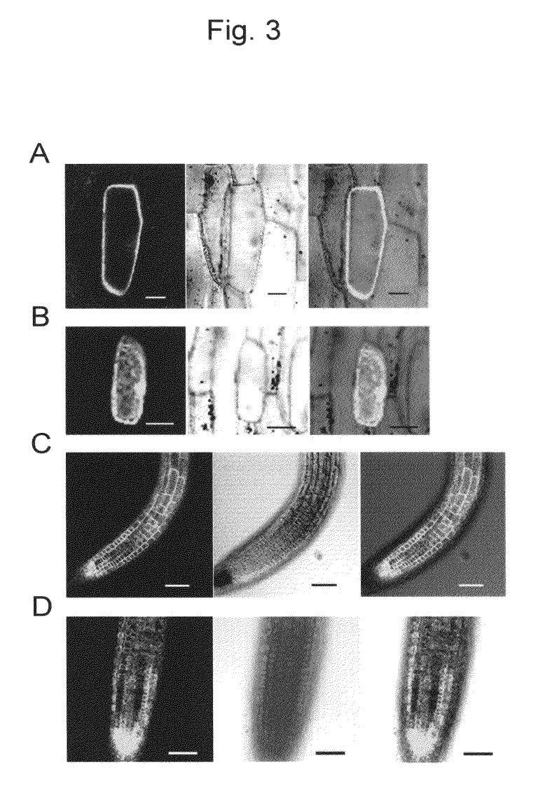

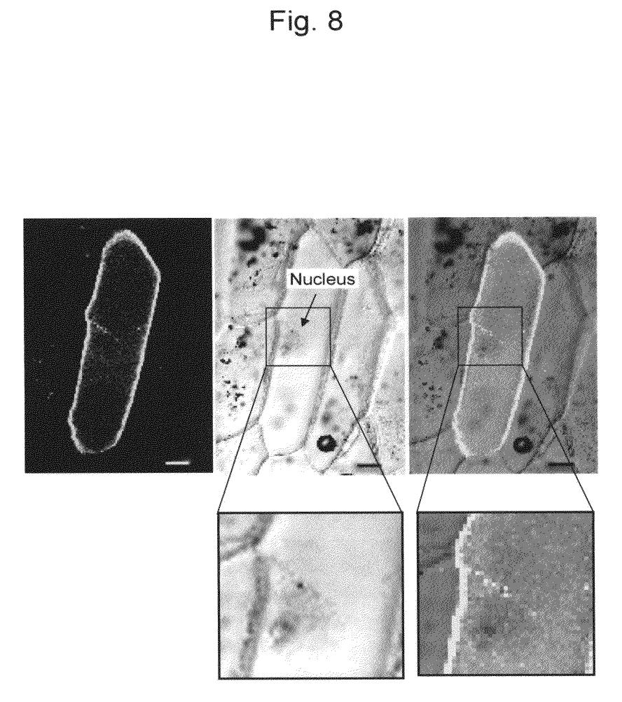

To examine the subcellular localization of the AtABCG25 protein, the present inventors constructed a vector for fusion of the AtABCG25 protein with a yellow fluorescent protein (YFP) produced under the control of the cauliflower mosaic virus (CaMV) 35S promoter. The gene-coding region (ORF) for AtABCG25 was placed downstream of 35S::YFP. The 35S::YFP-AtABCG25 recombinant gene was transiently expressed in onion epidermal cells by the particle bombardment method. Subcellular localization of the fusion protein was visualized by confocal imaging of the yellow fluorescent signals in the onion cells. The yellow fluorescence of the YFP-AtABCG25 recombinant protein was present around the cell surface in the onion epidermal cells (FIG. 3A and FIG. 8). However, signals of YFP alone as an experimental control was spread in the whole cell (FIG. 3B). Subsequently, wild-type Arabidopsis plants were transformed with the 35S::YFP-AtABCG25 recombinant vector. As with the results of the transient expression experiment, signals were observed on the cell surface of root tips in transgenic plants expressing YFP-AtABCG25 (FIG. 3C). Root tip cells do not contain large vacuoles (Shi, H. et al., 2002, Plant Cell 14: 465-477). The yellow fluorescence reflects localization of YFP-AtABCG25 to the plasma membrane but not in the tonoplast or cytoplasm. To exclude the possibility of localization of YFP-AtABCG25 to a cell wall, the root tip cells were observed after plasmolysis under highly osmotic conditions. The fluorescence in the root tip cells plasmolyzed by treatment with mannitol was observed apart from the cell wall (FIG. 3D). These results suggest that the AtABCG25 protein is a protein localized to plasma membrane.

### Functional Analysis of AtABCG25 Gene Product

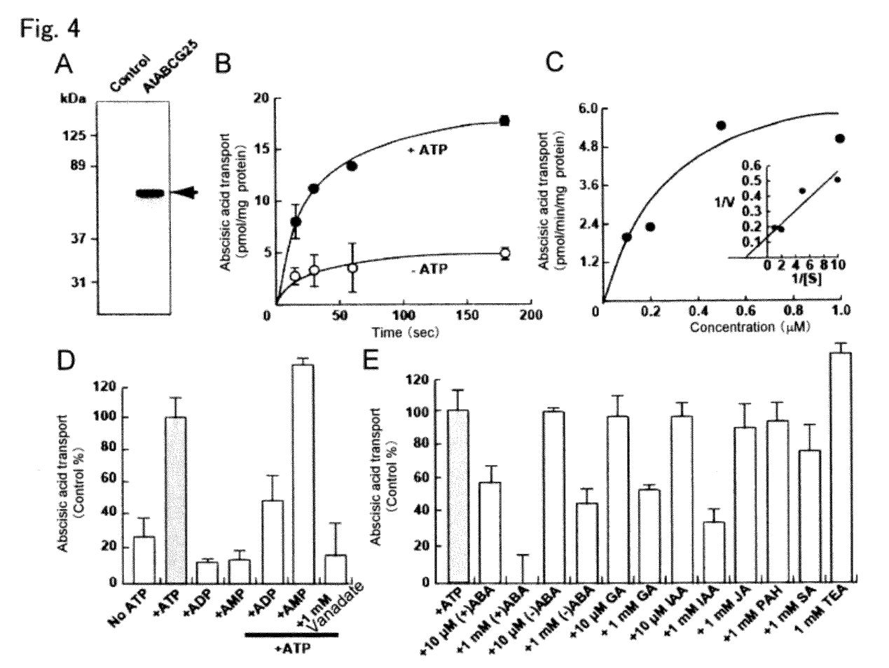

To pursue the possibility that AtABCG25 can transport ABA through the cell membrane, the present inventors attempted a vesicle transport assay. Since the regenerated membrane contains inside-out membrane vesicles, efflux activities can be detected as uptake signals. Vesicle membranes were prepared from Sf9 insect cells (Spodoptera frugiperda) transfected with a virus vector into which AtABCG25 cDNA had been integrated. The expression of the AtABCG25 protein was confirmed by Western blotting using an anti-AtABCG25 antibody (FIG. 4A). The present inventors found that the uptake of ABA labeled with a radioisotope was significantly promoted upon the addition of ATP (FIG. 4B). The ATP-dependent uptake of ABA exhibited saturation kinetics with Km and Vmax values of 230 nM and 6.2 μmol/min/mg protein, respectively (FIG. 4D). In contrast, neither ADP nor AMP promoted ABA uptake (FIG. 4D). Furthermore, ADP inhibited ATP-dependent ABA uptake, whereas AMP did not exhibit any inhibitory effect (FIG. 4D). Vanadate, which is an effective inhibitor of ABC transporters, also inhibited ATP-dependent ABA uptake (FIG. 4D). Cis-inhibition was performed to evaluate substrate specificity (FIG. 4E). The present inventors found that the ATP-dependent ABA uptake was inhibited by (+)ABA at a 10-fold concentration, but was not influenced by (−)ABA. Various phytohormones as well as anionic or cationic compounds exhibited no or substantially no inhibitory effect on ATP-dependent ABA uptake (FIG. 4E). Taken together, these results indicate that the AtABCG25 protein is responsible for ABA transport and that such protein acts on (+)ABA rather than (−)ABA.

### Overexpression of AtABCG25 and Its Effect on ABA Responsiveness

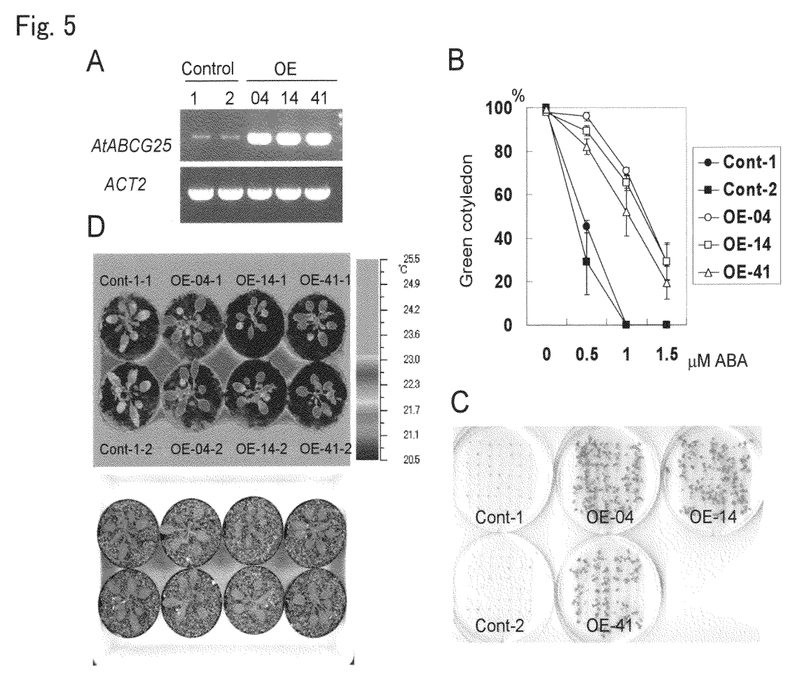

If AtABCG25 is an efflux factor in ABA transport, overexpression of AtABCG25 should influence ABA signaling. To evaluate this idea, the present inventors prepared transgenic Arabidopsis plants having the 35S::AtABCG25 construct vector (FIG. 5A). To examine ABA responsiveness, T3 seeds obtained from the resulting transgenic lines were tested for ABA inhibition of postgerminative growth. The ratio of the ABA inhibition of postgerminative growth was significantly reduced in 3 independent transgenic lines expressing the AtABCG25 transgene (FIGS. 5 B and C). This supports the hypothesis that AtABCG25 functions as an ABA efflux factor.

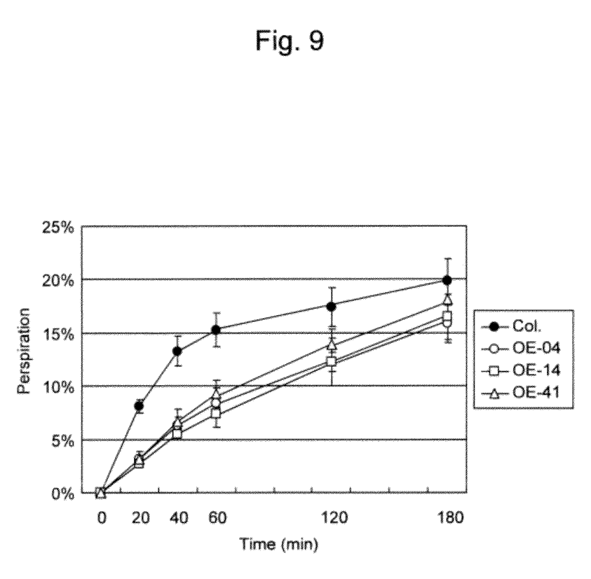

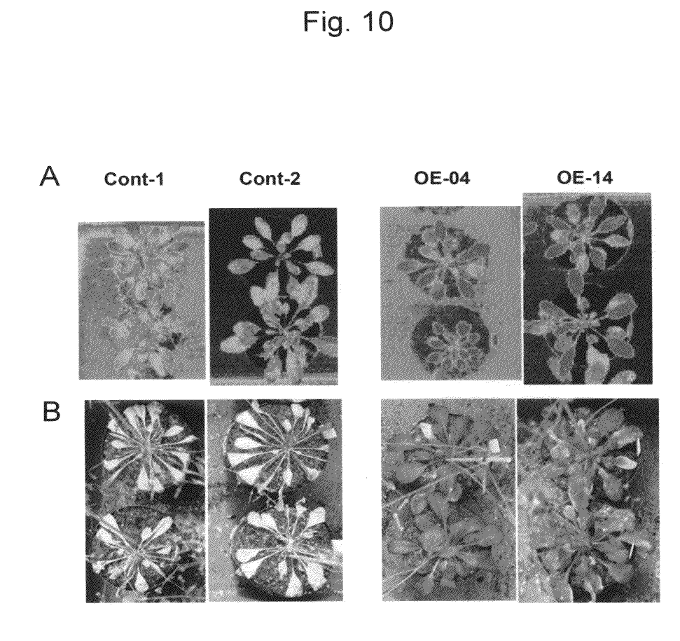

ABA acts directly on guard cells and induces stomatal closure (Schroeder, J. I. et al., 2001, Annu. Rev. Plant Physiol. Plant Mol. Biol. 52: 627-658). Thus, the present inventors further examined the aerial phenotypes related to stomatal regulation of plants overexpressing AtABCG25. As a result, the present inventors found that the leaf temperature of transgenic plants was higher than that of wild-type plants (FIG. 5D). This suggests that transpiration from the leaves of plants overexpressing AtABCG25 was suppressed. The present inventors also found that water loss from leaves detached from the transgenic plants was slower than that from leaves detached from wild-type plants (FIG. 9). Further, the present inventors conducted drought treatment and found that the growth rate after drought treatment of the plants overexpressing AtABCG25 (8 of 10 plants, 80.0%) was higher than that of control plants (1 of 6 plants, 16.7%) (FIG. 10). These results are consistent with the idea that AtABCG25 is an ABA transporter (exporter). It is possible that ABA is accumulated in the apoplastic area around guard cells in plants overexpressing AtABCG25.

### AtABCG25 is Transporter of ABA

In this study, the present inventors originally isolated atabcg25 mutants by screening for ABA sensitivity and found that AtABCG25 was expressed mainly in vascular tissues, which is the main area in which ABA is biosynthesized in plants (Cheng, W. H. et al., 2002, Plant Cell 14: 2723-2743; Koiwai, N. et al., 2004, Plant Physiol. 134: 1697-1707; Endo, A. et al., 2008, Plant Physiol. 147: 1984-1993). Further, they found that the fluorescent protein-fused AtABCG25 protein was localized to the plasma membrane in plant cells. Biochemical analyses indicated that AtABCG25 has the ability to transport ABA molecules. Additionally, plants overexpressing AtABCG25 were not sensitive to exogenous ABA at the seedling stage. Furthermore, plants overexpressing AtABCG25 had a higher leaf temperature and a lower rate of transpiration from detached leaves. This suggests that such factor influences stomatal regulation. These results demonstrate that AtABCG25 is considered to be one of the functional factors in the ABA transport mechanism and probably promotes the export of ABA through cell membranes from plant cells. Such findings reveal the presence of the ABA transport mechanism in plant cells and would give new insight to intercellular regulation of ABA transport in the ABA regulation networks.

In contrast to plants overexpressing AtABCG25, phenotypes in aerial organs, such as guard cells, were not observed in atabcg25 knockout mutant lines. The present inventors assumed that Arabidopsis has another factor that supplements the functions of AtABCG25. In addition to redundant genes, the combined actions of AtABCG25 and another half-molecule ABC transporter would be of particular interest because a half-molecule ABC transporter is known to work as a dimer complex (Samuels, L. et al., 2008, Annu. Rev. Plant Biol. 59: 683-707; Graf, G. A. et al., 2003, J. Biol. Chem. 278: 48275-48282). The results attained by the present inventors support the fact that AtABCG25 is one of the transporters functioning in ABA transport in Arabidopsis. ABA is an important phytohormone, which is thought to influence distant cells (Cheng, W. H. et al. 2002, Plant Cell 14: 2723-2743; Koiwai, N. et al., 2004, Plant Physiol. 134: 1697-1707; Endo, A. et al., 2008, Plant Physiol. 147: 1984-1993; Christmann, A., Weiler, E. W., Steudle, E., Grill, E., 2007, Plant J. 52: 167-174; Schachtman, D. P., Goodger, J. Q. D., 2008, Trends Plant Sci. 13: 281-287; Okamoto, M. et al., 2009, Plant Physiol. 149: 825-834), although any gene responsible for ABA transport has not been identified in any plant. The identification of AtABCG25 provides a clue to understanding of the ABA transport system in plants, and it provides new impetus for the study of ABA signaling between plant organs with regard to stress response or plant development.

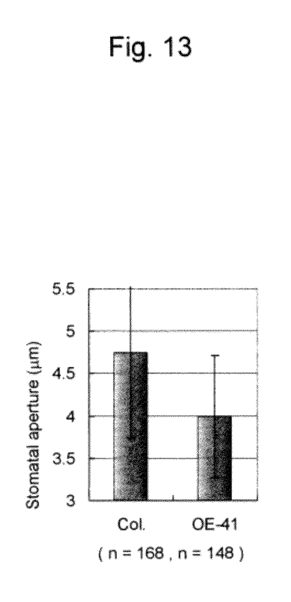

Further, experiments for supporting or reinforcing the above findings were carried out, and the results thereof are shown in FIGS. 13 to 15.

FIG. 13 shows the stomatal apertures (μm) of the rosette leaves of the 35S::AtABCG25 transgenic plant line (OE-41) and the control plant (Col.) (4-week-old each) measured using Suzuki's universal method of printing (SUMP). The results shown in the figure indicate that the stomatal aperture in the mature leaves of the plants overexpressing AtABCG25 is smaller than that in control plant.

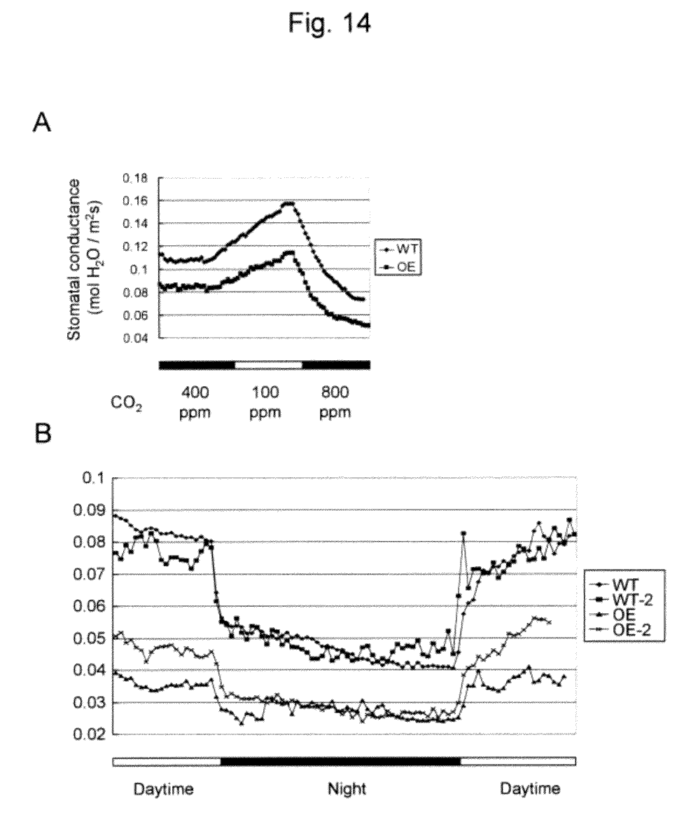

FIG. 14 shows that stomatal aperture of plants overexpressing AtABCG25 (OE) changes depending on CO₂ concentration and light/dark conditions, as with the case of wild-type plants (WT).

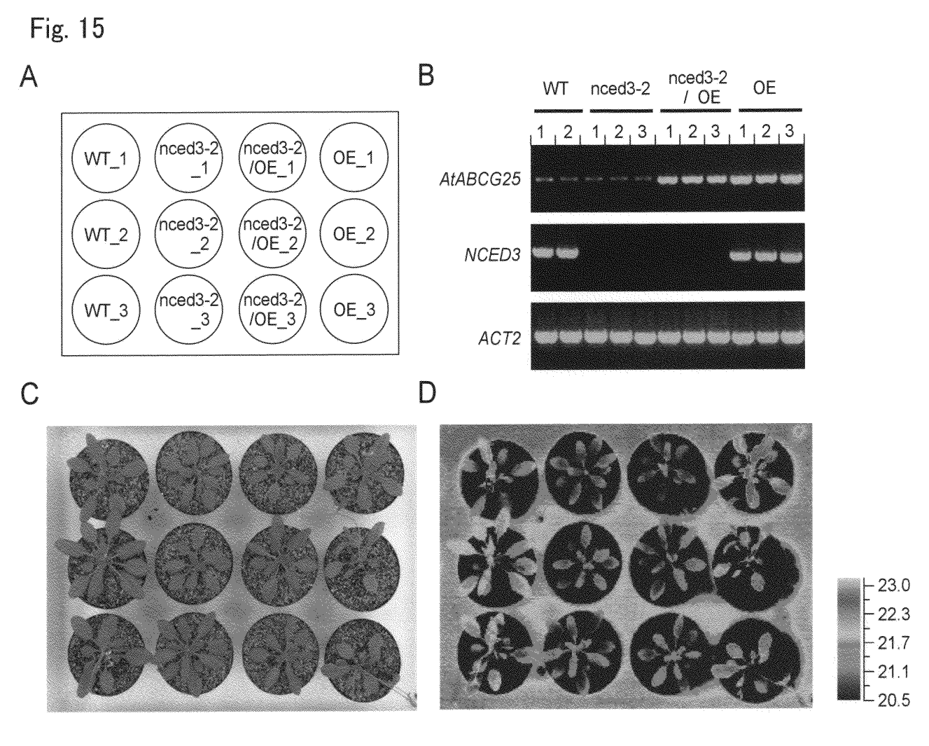

FIG. 15 shows data genetically verify that AtABCG25 is associated with the abscisic acid (ABA) signaling pathway. The experiments demonstrated that AtABCG25 is located downstream of NCED3 in the ABA signaling pathway.

It was demonstrated in the aforementioned Examples that tolerance to environmental stress can be imparted to a plant by overexpressing DNA comprising a nucleotide sequence encoding an exogenous ABA transporter protein in a plant mainly referring to Arabidopsis thaliana. However, transgenic plants of other plant species, including rice, with similar effects can also be easily produced according to the methods described in the description and the Examples.

### INDUSTRIAL APPLICABILITY

The present invention provides environmental stress-tolerant plants, and it is thus applicable in industrial fields, particularly in agricultural, forestry, paper manufacturing, and other industries.

### SEQUENCE LISTING FREE TEXT

SEQ ID NOs 3 to 15: primers
SEQ ID NOs 16 to 18: synthetic peptides

All publications, patents, and patent applications cited herein are incorporated herein by reference in their entirety.

1. A transgenic plant tolerant to environmental stress, which comprises DNA encoding an exogenous abscisic acid (ABA) transporter protein in an expressible manner, wherein the ABA transporter protein is a protein having biological activity of exporting ABA from a cell through a cell membrane.

2. The transgenic plant according to claim 1, wherein the DNA encoding the ABA transporter protein is any of polynucleotides (DNAs) (a) to (d) below:
(a) DNA comprising a nucleotide sequence encoding a protein comprising the amino acid sequence from Arabidopsis thaliana as shown in SEQ ID NO: 2 or the amino acid sequence from rice as shown in SEQ ID NO: 20;
(b) DNA comprising a nucleotide sequence encoding an amino acid sequence of a homolog of the protein as recited in (a), which is derived from a plant other than the plant as recited in (a) and has ABA transport activity;
(c) DNA comprising a nucleotide sequence encoding an amino acid sequence having 70% or higher identity with the amino acid sequence as shown in SEQ ID NO: 2 or 20 or an amino acid sequence of the homolog and having ABA transport activity; and
(d) DNA comprising a nucleotide sequence encoding an amino acid sequence having substitution, deletion, or addition of one or a plurality of (and preferably 1 or several) amino acids in the amino acid sequence as shown in SEQ ID NO: 2 or 20 or an amino acid sequence of the homolog and having ABA transport activity.

3. The transgenic plant according to claim 2, wherein DNA encoding a protein comprising the amino acid sequence as shown in SEQ ID NO: 2 or 20 comprises an ABA transporter protein-encoding sequence as shown in SEQ ID NO: 1 or 19, respectively.

4. The transgenic plant according to claim 1, wherein the environmental stress tolerance is drought stress tolerance.

5. The transgenic plant according to claim 1, wherein the plant is a dicotyledonous or monocotyledonous plant.

6. A progeny of the transgenic plant according to claim 1, which has environmental stress tolerance.

7. A cell, tissue, or seed of the transgenic plant according to claim 1 or the progeny of the transgenic plant which has environmental stress tolerance.

8. A method for producing a transgenic plant tolerant to environmental stress that comprises DNA comprising a nucleotide sequence encoding an exogenous abscisic acid (ABA) transporter protein in an expressible manner, comprising the steps of:
introducing the DNA into a plant cell or callus so that the DNA can be expressed therein; and
regenerating a plant body from the plant cell or callus,
wherein the ABA transporter protein has biological activity of exporting ABA from a cell through a cell membrane.

9. A method for imparting environmental stress tolerance to a plant, comprising the steps of:
introducing into a plant or its cell DNA comprising a nucleotide sequence encoding an exogenous ABA transporter protein so that the plant or the cell comprises the DNA in an expressible manner; and
thereby imparting environmental stress tolerance to the plant,
wherein the ABA transporter protein has biological activity of exporting ABA from a cell through a cell membrane.

10. The method for imparting environmental stress tolerance to a plant, comprising the steps of:
introducing into a plant or its cell DNA comprising a nucleotide sequence encoding an exogenous ABA transporter protein so that the plant or the cell comprises the DNA in an expressible manner; and
thereby imparting environmental stress tolerance to the plant,
wherein the ABA transporter protein has biological activity of exporting ABA from a cell through a cell membrane, wherein the DNA is as defined in claim 2.
